(19) United States
(12) Patent Application Publication
(10) Pub. No.: US 2024/0008358 A1
(43) Pub. Date: Jan. 4, 2024

(54) TRIPLET QUENCHING

(71) Applicant: THE UNIVERSITY OF QUEENSLAND, St Lucia, Queensland (AU)

(72) Inventors: Van T. N. MAI, St Lucia, Queensland (AU); Shih-Chun LO, St Lucia, Queensland (AU); Viqar AHMAD, St Lucia, Queensland (AU); Jan SOBUS, St Lucia, Queensland (AU); Ebinazar B. NAMDAS, St Lucia, Queensland (AU)

(21) Appl. No.: 18/247,963

(22) PCT Filed: Oct. 6, 2021
(86) PCT No.: PCT/AU2021/051165

§ 371 Date: Apr. 5, 2023

(30) Foreign Application Priority Data
Oct. 6, 2020 (AU) 2020903603

(51) Int. Cl. H10K 85/60; C07D 209/86

(52) U.S. Cl. CPC H10K 85/625; C07D 209/86; H10K 85/1135; C07C 2603/18; H10K 85/6572

(57) ABSTRACT

The disclosure relates to a substituted cyclooctatetraene triplet excited-state quencher compound of Formula (I): wherein: Z is a wide band gap moiety; L is a non-conjugating linker group; each R, which may be the same or different, is a non-conjugating substituent; n is an integer from 0 to 7; and m is an integer from 1 to 6. The disclosure further relates to use of such compounds as triplet quenchers, compositions comprising such compounds, films or coatings comprising said compounds or compositions, and use of said compositions or films or coatings as active gain media for light amplification.

### FIELD OF THE INVENTION

The present invention relates to novel solid-state triplet quenchers suitable for use in quenching for both optical and electrical excitations.

### BACKGROUND OF THE INVENTION

The discovery of the first laser in 1960 has opened up a wide variety of applications, ranging from fundamental usages, such as scientific optical excitation and photolithography, to industrial laser cutting, drilling, military applications, medical imaging and surgery. Compared to their inorganic counterparts, organic lasers offer many advantages such as compact size, high mechanical flexibility, high transparency and high wavelength tunability. Moreover, among organic laser dyes, solution-processable dyes offer additional advantages of employing low cost and large-area manufacturing techniques such as spin-coating or ink-jet printing in device fabrication.

Organic lasers require a secondary excitation source such as gas lasers, inorganic solid state lasers or light-emitting diodes (LEDs) for optical excitation. Direct electrical excitation of organic lasers, however, is significantly more challenging.

Current driven organic semiconductor laser diodes (OSLDs) have been demonstrated (Sandanayaka, A. S. D. et al. Appl. Phys. Express 2019, 12, 0610102019). However, the current threshold is still extremely high with a value of about 1000 A cm⁻². This high current density inevitably creates a major issue for OSLDs, that is high accumulation of triplet excitons, leading to significant losses such as triplet absorption, singlet-triplet annihilation (STA) and triplet-polaron annihilation (TPA). Given that in optical excitation there is only a small fraction of triplets converted from singlets via intersystem crossing, it is important to note that in electrical excitation, 75% of excitons generated are triplets according to spin statistics. This means that the large proportion of the non-emissive triplet excited state together with its significantly long lifetime (often in microsecond range) leads to fast triplet accumulation in electrical excitation, compared to singlet excited state (in ns range). These non-emissive triplet excited-states bring about above-mentioned losses, or even result in no laser activity. Moreover, these non-emissive triplet excited-states can cause stability issues. As a result, lasing thresholds under electrical excitation are significantly higher than those under optical excitation. Therefore, the ability to manage the non-emissive triplet states is crucial for the improvement of OSLDs.

Approaches to reduce the triplet excited-state population have been reported by using different triplet excited-state quenchers (TSQs). Among these TSQs, the most efficient quenchers are oxygen, anthracene and cyclooctatetraene (COT). However, all of these TSQs have their respective limitations that render them incompatible with OSLD devices.

Specifically, due to the unique triplet ground state, molecular oxygen is easily converted into reactive singlet oxygen species, which is detrimental to the active organic semiconductor materials in the devices due to photo-oxidation and photo-degradation, in addition to the undesired singlet quenching. Anthracene and its derivatives are known to have relatively long triplet excited-state lifetimes (≈20 ms), where accumulation of triplets on the anthracene molecules is the key issue and source of another triplet accumulation. In contrast, COT has been highlighted as a more promising TSQ candidate in view of its considerably shorter triplet excited-state lifetime (100 μs) and low triplet energy without oxidation of organic laser dyes. Unfortunately, COT has a melting point of around −5 to −3° C. and is a volatile liquid at ambient conditions. Hence, it is not compatible with thin film devices and it has only been possible to demonstrate utility of COT for liquid-state organic dye lasers.

There is a need for improved triplet excited-state quenchers for solid-state laser applications that address one or more disadvantages of current quenchers.

### SUMMARY OF THE INVENTION

The present inventors have discovered a class of novel triplet excited-state quenchers that can address one or more of the disadvantages of known triplet excited-state quenchers. In particular, the inventors have discovered that these solid-state triplet excited-state quenchers can function under optical or electrical excitations. As such, the compounds of the invention can find application as triplet quenchers in the field of organic lasers, for example, organic semiconductor laser diodes.

Accordingly, in a first aspect there is provided a compound of Formula (I):

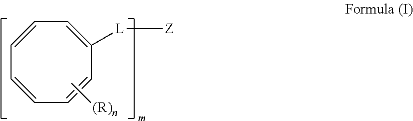

wherein:
Z is a wide band gap moiety;
L is a non-conjugating linker group;
each R, which may be the same or different, is a non-conjugating substituent;
n is an integer from 0 to 7; and
m is an integer from 1 to 6.

The compounds of Formula (I) have application as triplet excited-state quenchers. The Z substituent is a moiety derived from a corresponding wide band gap material. Preferably, the wide band gap material has a high singlet and triplet energy. This may be provided by a group such as, but not limited to, alkyl, aryl, amino, amide, ester, hydroxy, nitro, heteroaryl, nitrile, or carboxylic acid. In some embodiments, the wide band gap moiety is a wide band gap host moiety. In some embodiments, the wide band gap moiety may contain a substituent with a low ionisation potential or a high electron affinity, or a substituent with both low ionisation potential and high electron affinity species. Suitably substituent Z is attached to the linker group through a covalent bond. Preferably the Z substituent is attached to the linker through a C atom or an N atom in the structure of the Z moiety in such a manner that the photophysical or electronic properties of the parent host material are substantially preserved in the Z substituent. Suitably, the cyclooctatetraene (COT) moiety is covalently attached to the linker group L. Preferably the linker group does not form conjugation with either the Z moiety or the COT moiety, such that the photophysical or electronic properties of the COT moiety and the Z moiety are substantially the same as the parent COT molecule and the parent wide band gap material.

In some embodiments, the compound of Formula (I) is a compound of Formula (Ia):

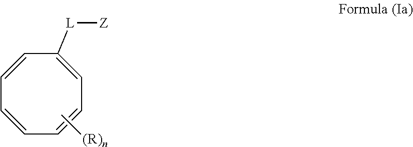

wherein:
Z is a wide band gap moiety;
L is a non-conjugating linker group;
each R, which may be the same or different, is a non-conjugating substituent; and
n is an integer from 0 to 7, preferably 0 to 3.

In some embodiments, the compound of Formula (I) or (Ia) is a compound of Formula (Ib):

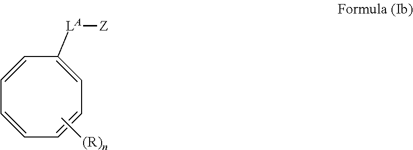

wherein:
Z, R and n are as defined above for a compound of Formula (I); and
L^{A} is a branched or straight chain alkylene linking group comprising two or more carbon atoms; or
L^{A} is —X¹-L^{b}-X²—;
wherein L^{b} is a branched or straight chain alkylene group comprising two or more carbon atoms and X¹ and X² are independently selected from an ether, amino, amide or ester group; and wherein one of X¹ and X² may be absent.

In some embodiments, preferably n is 0 and the compound of formula (I), (Ia) or (Ib) is a compound of Formula (Ic):

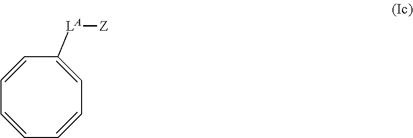

wherein:
Z and L^{A} are as defined above for a compound of Formula (Ib).

In some embodiments, Z is a moiety derived from mCP, CBP, A, CZ, Q, or Pz. In some embodiments, Z is an mCP [1,3-bis(N-carbazolyl)phenyl]; a CBP [4,4′-bis(N-carbazolyl)-1,1′-biphenyl]; an A [anthracenyl]; a Cz [carbazolyl]; a Pz [phenoxazinyl], or a Q [quinolinyloxy] moiety. In some embodiments, Z is a Ph [phenyl]; a BP [biphenyl]; a TP [tetraphenyl]; a PP [polyphenylenyl]; a TT [triptycenyl]; an AM [adamantanyl]; a TPA [triphenylamino]; or a TPM [tetraphenylmethane].

In some embodiments, Z is an mCP moiety, i.e., 1,3-bis(N-carbazolyl)phenyl and the compound of formula (I), (Ia), (Ib) or (Ic) is a compound of Formula (Id):

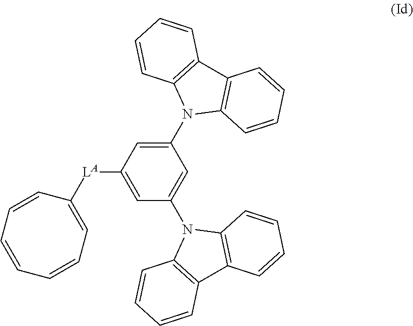

wherein L^{A} is a branched or straight chain alkylene linker, for example a straight chain or branched chain alkylene linker with two or more carbon atoms. A compound of Formula (Id) is herein referred to as mCP-Cn-COT.

In some embodiments, the compound of Formula (I), (Ia), (Ib), (Ic) or (Id) is mCP-C6-COT:

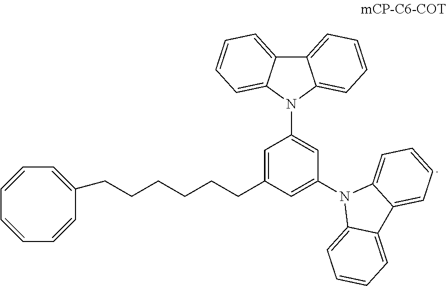 [SMILES: C1=C\C=C/C(CCCCCCc2cc(-n3c4ccccc4c4ccccc43)cc(-n3c4ccccc4c4ccccc43)c2)=C\C=C/1]

In some embodiments, Z is a moiety derived from phenyl, biphenyl, pyridine, oxadiazole, imidazole, pyrimidine, triazine, bipyridine, phenanthroline, benzothiadiazole, perylenediimide, benzoisoquinolinedione, quinoline, quinoxaline, arylphosphine oxide, indigo, perfluoroarene, arylborane, (di)cyanopyrazine, (di)cyanoquinoxaline, or dioxidethioxanthenone.

In some embodiments, the compound of Formula (I) is a compound of Formula (Ie):

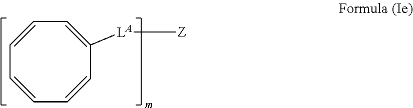

wherein:
Z is as defined above for a compound of Formula (I); and
L^{A} is a branched or straight chain alkylene linking group comprising two or more carbon atoms; or
L^{A} is —X¹-L^{b}-X²—;
wherein L^{b} is a branched or straight chain alkylene group comprising two or more carbon atoms and X¹ and X² independently represent an ether, amino, amide or ester group; wherein one of X¹ and X² may be absent; and m is an integer from 1-4.

In some embodiments, the compound of Formula (I) is FI-COT:

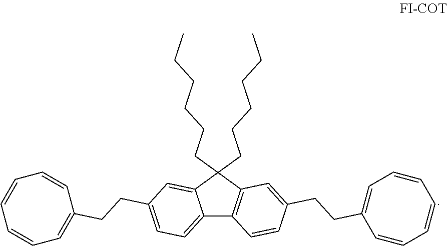 [SMILES: CCCCCCC1(CCCCCC)c2cc(CCC3=C/C=C\C=C/C=C\3)ccc2-c2ccc(CCC3=C/C=C\C=C/C=C\3)cc21]

In some embodiments, a non-conjugating substituent R is a group -L-Z, wherein L and Z are as hereinbefore defined, such that the COT group is linked to more than one -L-Z moiety. In some embodiments, the compound of Formula (Ia) is a compound of Formula (If):

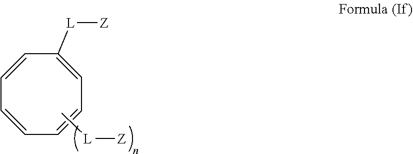

wherein L and Z are as defined above for compounds of Formula (I) or (Ia) and n is an integer from 1 to 3. In some embodiments, L is a linker L^{A} as described herein.

In at least one embodiment, the compounds of Formula (I) find application as a triplet quencher. Accordingly, in another aspect, there is provided a use of a compound of Formula (I) as a triplet quencher.

In another aspect of the invention, there is provided a composition comprising a compound of Formula (I) and an organic semi-conductor laser dye. In some embodiments, the laser dye is BSBCz-EH or BSBCzCN-EH.

In some embodiments, the composition as described herein is provided as a coating or a thin film, optionally the coating or thin film is provided on a substrate.

In yet another aspect, the present invention provides a use of a compound as described herein as a triplet excited-state quencher. In some embodiments, the compound is a triplet excited-state quencher for use in organic solid-state lasers. In some embodiments, the triplet excited-state quencher is for use in at least one of: organic solid-state lasers; opto-electronic applications; laser diodes such as organic semiconductor laser diodes; light-emitting diodes; solar cells; sensors; and photorefractive devices.

In another aspect, there is provided a composition as described herein as an active gain medium for light amplification, for example for light amplification in organic solid-state lasers. In some embodiments, the composition is for use in at least one of: organic solid-state lasers; opto-electronic applications; laser diodes; light-emitting diodes; solar cells; sensors; and photorefractive devices. In some embodiments, the laser is electrically pumped.

### BRIEF DESCRIPTION OF THE DRAWINGS

FIG. 1: Cyclic voltammograms of mCP-C6-COT; quoted against ferrocenium/ferrocene (Fc⁺/Fc) couple; showing oxidation in dichloromethane (red solid line) and reduction in tetrahydrofuran (blue solid line) (1 mM). Differential pulse (DP) voltammetry was also conducted for reduction of mCP-C6-COT in tetrahydrofuran (dotted blue line). Measurements were conducted with 0.1 M tetra-n-butylammonium perchlorate electrolyte; working electrode=glassy carbon; reference electrode=0.01 M AgNO₃ in acetonitrile; counter electrode=platinum; scan rate=100 mV s⁻¹.

FIG. 2: Solution absorption and normalised photoluminescence (PL, solid lines) spectra of COT, mCP and mCP-C6-COT in toluene (inset shows the weak COT absorption). Excitation wavelength=290 nm. At this low temperature, phosphorescence seen as peaks from 400 to 500 nm in mCP (yellow highlights) are not observed in mCP-C6-COT, indicating efficient quenching of the triplet excited state of the mCP moiety within mCP-C6-COT. Excitation wavelength=300 nm.

FIG. 3: TCSPC PL decay curves for mCP and mCP-C6-COT in toluene.

FIG. 4: Transient absorption spectra and decay kinetics at 400 and 617 nm for mCP in ambient conditions.

FIG. 5: Results of transient absorption spectroscopy. Two distinct transient absorption bands obtained in case of (a) mCP under ambient conditions (aerated) (b) mCP under degassed condition (deoxygenated) and (c) mCP-COT (under degassed conditions). d Comparison of triplet excited-state absorption decay (at 400 nm) under ambient and degassed conditions in case of mCP.

FIG. 6: Film absorption and PL spectra of BSBCz-EH for varying concentrations of mCP-C6-COT. (a) Absorption spectrum of BSBCz-EH neat and blend films with varying mCP-C6-COT doping concentrations (1 wt %, 3 wt %, 5 wt %, 10 wt %, 20 wt %, 50 wt % and 90 wt %), inset shows chemical structure of the solution-processable blue-emitting BSBCz-EH dye. Excitation wavelength=380 nm. (b) Normalised PL intensity (bottom) spectra of BSBCz-EH neat and blend films with varying mCP-C6-COT doping concentrations (1 wt %, 3 wt %, 5 wt %, 10 wt %, 20 wt %, 50 wt % and 90 wt %).

FIG. 7: Transient PL. Characteristics of encapsulated BSBCz-EH neat film and blend films with different mCP-C6-COT blending concentrations (5 wt %, 10 wt %, and 20 wt %), which can be compared with its blend film with 20 wt % in mCP. Excitation wavelength=355 nm; laser beam excitation power=2.65 mW; pulse width=200 μs and pulse interval=10 ms.

FIG. 8: Relative drop in STA as a function of mCP-C6-COT and ADN quencher concentrations for BSBCz-EH and Alq3/DCM2, (Zhang, Y. & Forrest, S. R. Phys. Rev. B 2011, 84, 241301), respectively. In the absence of STA, there would be no singlet-triplet interaction between populations; therefore, the singlet population should shortly (under 1 μs) saturate at a steady value where the positive pumping term is balanced out by negative fluorescent ISC and SSA terms (assuming positive contribution of TTA to be negligible) and there is no impact of growing triplet population. Since the singlet population directly correlates to the light intensity, one can treat the difference between peak and steady state in a neat film as a total (i.e., 100%) loss due to STA in a system without triplet quencher. Then, the relative decrease in STA plotted against the quencher concentration can be a measure of how successful the triplet manager is in the system.

FIG. 9: Photostability of BSBCz-EH. PL intensity/initial PL intensity (I/I₀) of a BSBCz-EH neat film (green line), a blend film with 20 wt % mCP (pink line), and a blend film with 20 wt % mCP-C6-COT (violet line) measured under CW photoexcitation with a power of 200 mW cm⁻² at 405 nm. Excitation area=2.5 mm×2.5 mm circle.

FIG. 10: ASE thresholds with varying concentrations of mCP-C6-COT. Comparable ASE thresholds were achieved in BSBCz-EH neat and blend films with mCP-C6-COT at 5 wt %, 10 wt % and 20 wt % blend concentrations.

FIGS. 11(a)-(d): ASE thresholds of neat and blend films. Comparable ASE thresholds were achieved in BSBCz-EH (a) neat film and blend film with (b) 5 wt %, (c) 10 wt % and (d) 20 wt % of mCP-C6-COT. ASE thresholds were estimated from the abrupt change in the slope of input-output intensity (in logarithmic-logarithmic scale) together with significant decrease in full-width at half-maximum (FWHM) (left); photoluminescence spectra at excitation powers below and above ASE threshold showing spectral narrowing with increasing pump intensities (right).

FIG. 12: Neat and blend-film PLQYs of CBP, BSBCz-EH and BSBCz-CN-EH with various mCP-C6-COT doping concentrations (i.e., 0%, 1 wt %, 3 wt %, 5 wt %, 10 wt %, 20 wt %, 50 wt % and 90 wt %). Excitation wavelength=330 nm for CBP, 380 and 340 nm for BSBCz-EH, and 410 and 330 nm for BSBCz-CN-EH. The blue and green squares (at top right corner) show blend-film PLQYs of 5 wt % BSBCz-EH and BSBCz-CN-EH in CBP, respectively.

FIGS. 13(a)-(d): Brightness and EQE comparison of neat and mCP-C6-COT blend small-area OLEDs for pulse inputs. (a) A comparison of EL intensity of the neat and blend BSBCz-EH OLEDs at a current density of 50 A cm⁻². (b) Normalised EL intensities, showing a significant STA in the neat device and substantial reduction of the same in blend. (c) EQE versus current density for neat and blend devices at 50 A cm⁻². (d) Brightness versus current density plot for neat and blend devices. OLED area=0.2 mm²; pulse widths=100 ns.

### DETAILED DESCRIPTION OF THE INVENTION

### Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much as 10, 9, 8, 7, 6, 5, 4, 3, 2 or 1% to a reference quantity, level, value, number, frequency, percentage, dimension, size, amount, weight or length. The term “approximately” is construed similarly.

When used herein the terms “w/w %”, “w/v %” and “v/v %” mean, respectively, weight to weight, weight to volume, and volume to volume percentages.

As used herein, the term “and/or” refers to and encompasses any and all possible combinations of one or more of the associated listed items, as well as the lack of combinations when interpreted in the alternative (or).

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. Thus, the use of the term “comprising” and the like indicates that the listed integers are required or mandatory, but that other integers are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Each embodiment described herein is to be applied mutatis mutandis to each and every embodiment unless specifically stated otherwise.

### Abbreviations

As used herein the symbols and conventions used in these processes, schemes and examples are consistent with those used in the contemporary scientific literature, for example, the Journal of the American Chemical Society. Specifically the following abbreviations may be used in the specification:

ASE (amplified spontaneous emission); HOMO (the highest occupied molecular orbital); LUMO (the lowest unoccupied molecular orbital); OLED (organic light-emitting diode); OSLD (organic semiconductor laser diode); organic solid-state laser (OSSL); PL (photoluminescence); nanosecond (ns); PLQY (photoluminescence quantum yield); STA (singlet-triplet annihilation); TPA (triplet-polaron annihilation). TAS (transient absorption spectroscopy); TCSPC (time correlated single photon counting); TSQ (triplet excited-state quencher); AND (9,10-di(naphth-2-yl)anthracene); BSBCz (4,4′-bis[(N-carbazole)styryl]biphenyl); COT (cyclooctatetraene); HBT (2-hydroxyphenylbenzothiazole); IPA (isopropanol, 2-propanol); mCP (1,3-bis(N-carbazolyl)benzene); DCM (dichloromethane); DMF (N,N′-dimethylformamide); DMSO (dimethyl sulfoxide); 9-BBN (9-borabicyclo[3.3.1]nonane); MIBK(methyl isobutyl ketone, 4-methyl-2-pentanone); THF (tetrahydrofuran).

### Compounds of the Invention

The compounds of the invention are based on the combination of a wide band gap moiety and a 1,3,5,7-cyclooctatetraene (COT) moiety. The compounds thus comprise a COT moiety and wide band gap moiety joined by a linker group, preferably the two moieties are covalently joined. The wide band gap moiety comprises a wide gap functional group, preferably with a high singlet and triplet energy. The inventors have discovered that in several embodiments the compounds of the invention are useful as solid-state triplet excited-state quenchers (TSQ) under optical or electrical excitations in the nanosecond (ns) range. These triplet excited-state quenchers are in a solid form under ambient conditions. In addition, they typically have good solubility in common organic solvents, thus providing access to solution processability. As such, the compounds of the invention find application as triplet quenchers in organic lasers, for example, organic semiconductor laser diodes.

Compounds of the invention are represented by the generic Formula (I):

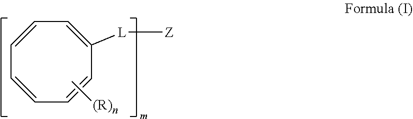

wherein:
Z is a wide band gap moiety;
L is a non-conjugating linker group;
each R, which may be the same or different, is a non-conjugating substituent;
n is an integer from 0 to 7; and
m is an integer from 1 to 6.

In some embodiments, m is 1, 2, 3 or 4. In some embodiments, m is 1 or 2. In some embodiments n is 0, 1, 2 or 3. In some embodiments, n is 0.

The wide band gap moiety Z is derived from a material comprising a group with a large band gap. Such large band gap groups include, but are not limited to, alkyl, aryl, amino, amide, ester, hydroxy, nitro, heteroaryl, nitrile, and carboxylic acid groups. In preferred embodiments, the wide band gap material has a high singlet and triplet energy. Examples of wide band gap materials are well known to the skilled person and such examples include carbazolyl, anthracenyl, imidazolyl, phenoxazinyl, quinolinyloxy and host materials. Examples include fluorescent or phosphorescent host materials. Suitable materials are described in, for example, Chaskar, A. et al., Adv. Mater. 2011, 23, 3876; Tao, Y. et al., Chem. Soc. Rev., 2011, 40, 2943; and Yook, K. S. & Lee. J. Y., Adv. Mater. 2012, 24, 3169.

Examples of phosphorescent wide band gap host materials are well known in the art, and include, but are not limited to, commercially available materials. For example, suitable host materials include those available from Luminescence Technology Corp (Lumtec), Taiwan (https://www.lumtec.com.tw/products.php?sn=42).

Examples of phosphorescent host materials include: 4-CbzBiz (9-(1,2-diphenyl-1H-benzo[d]imidazol-4-yl)-9H-carbazole); CzSi [9-(4-tert-butylphenyl)-3,6-bis(triphenylsilyl)-9H-carbazole]; BCzPh [9,9′-diphenyl-9H,9′H-3,3′-bicarbazole]; m-BPySCZ (5-(3-(9H-carbazol-9-yl)-phenyl)-3-(pyridine-3-yl)pyridine); m-DBPPO; TCPY (9,9′,9″-(pyridine-2,4,6-triyltris(benzene-3,1-diyl))tris(9H-carbazole)]; PFN-B; oCzTP 9 9,9′-(2-([1,2,4]triazolo[1,5-a]pyridin-2-yl)-1,3-phenylene)bis(9H-carbazole)]; m-POPyCz [(5-(9′H-[9,3′:6′,9″-tercarbazol]-9′-yl)pyridin-3-yl)diphenylphosphine oxide]; NzmPy2Cz; Tri-o-2PO; PO-T2T (2,4,6-tris[3-(diphenylphosphinyl)phenyl]-1,3,5-triazine]; o-CBP [2,2′-di(9H-carbazol-9-yl)biphenyl]; CzppQ [9-(4-(4-phenylquinolin-2-yl)phenyl)-9H-carbazole]; BPOBP [1,3-bis(3-(diphenylphosphoryl)phenyl)benzene]; POCz3 [3,3′,3″-phosphinylidynetris[9-phenyl-9-carbazole]; BCzTPA [4,4′-(9H,9′H-3,3′-bicarbazole-9,9′-diyl)bis(N,N-diphenylaniline)]; POBPmDPA [2′-(diphenylphosphinyl)-N,N-bis(4-methylphenyl)-1,1′-biphenyl]-2-amine]; BBICT; m-cbtz; CbBPCb; BCzSCN; DTP-mCP; STDBT-4; ABBP; CzTP; PPO21 [3-(diphenylphosphoryl)-9-(4-(diphenylphosphoryl)phenyl)-9H-carbazole]; SiMCP2; SPPO1; 35DCZPPy; EFIN; CzSi; BSB [4,4′-di(triphenylsilyl)-biphenyl]; BST; DOFL-CBP; MPMP; UGH-3; UGH-2; Spiro-2CBP; FL-2CBP; DPFL-CBP; Spiro-CBP; DMFL-CBP; CDBP; CBP [4,4′-bis(carbazol-9-yl)biphenyl]; TcTa; TCP; MCP; 46DCzPPM; CPCBPTz; PVK; PYD-2Cz; pBCb2Cz; m-CBP; DV-CBP; 2,7-F-PVF; DPEPO; BTP1; PCz-BFP; DBFPPO; DFCzPO; SPPO11; TSPO1; mCPPO1; CNBzIm; Ph-MCP; G3-tCbz; CzPO2; PCMO; NP4PPO; NP3PPO; Dspiro-PO; SPPO21; PFN-DOF; PP027; POAPF; BCPO; DCB; SPPO13; TCz1; UGH-1; FATPA; SimCP; and 26DCzPPY. In some embodiments, the Z moiety in a compound of Formula (I) is derived from a wide band gap phosphorescent host material that is non-polymeric.

In some embodiments, the Z moiety is derived from a fluorescent host material. Such hosts are well known in the art and include, but are not limited to, substituents based on the structure of commercially available host materials such as those available from Lumtec (https://www.lumtec.com.tw/products.php?sn=42).

Examples of fluorescent wide band gap host materials include BH-9PA; pDPFB; SF34; MAD-1N; o-CBP; TCPZ; POPH; DPTPCz; BCz-Si; 3CzPFP; 4ICDPy; SSTF; POSTF; SF3PO; CPPyC; Znq2; 6FAlq3; ADP; BAnF8Pye; DAn6FPye; BAnFPye; DBP; BUBH-3; 4P-NPB; BANE; TPyPA, DMPPP; DMP; DBPenta; m-Bpye; p-Bpye; Spiro-pye; TPBA; BPPF; TPB3; 2,2′-Spiro-pye; BDAF; TSBF; BSBF; MADN; TDAF; p-DMDPVBi; DPVBi; TBADM; ADN; Alq3 and SF4. In some embodiments, the Z moiety in a compound of Formula (I) is derived from a wide band gap fluorescent host material that is non-polymeric.

In some embodiments, the Z substituent is Cz (carbazolyl); A (anthracenyl); Pz (phenoxazinyl); Q (quinolinyloxy); CBP [4,4′-bis(carbazol-9-yl)biphenyl]; or mCP [1,3-bis(N-carbazolyl)phenyl]. In some embodiments, the substituent is carbazol-9-yl. In some embodiments, the Z substituent is a moiety derived from mCP [1,3-bis(N-carbazolyl)phenyl], i.e., Z is 1,3-bis(carbazol-9-yl)phenyl. In some embodiments, Z is a moiety derived from a wide band gap material such as benzene, biphenyl, tetraphenyl, polyphenylenyl, triptycenyl, adamantane, triphenylamine, or tetraphenylmethane. In some embodiments, the Z moiety is derived from an aryl molecule such as 9,9-dihexyl-9H-fluorene and is preferably linked to two COT moieties via linking groups.

In some embodiments, Z is a moiety derived from pyridine, oxadiazole, imidazole, pyrimidine, triazine, bipyridine, phenanthroline, benzothiadiazole, perylenediimide, benzoisoquinolinedione, quinoline, quinoxaline, arylphosphine oxide, indigo, perfluoroarene, arylborane, (di)cyanopyrazine, (di)cyanoquinoxaline, or dioxidethioxanthenone.

It will be appreciated that, in the compounds of Formula (I), the wide band gap moiety may be attached to the linker through any suitable atom in the wide band gap moiety provided that the linkage is chemically correct and atom valencies are satisfied. It will be understood that the structure of a host material may have more than one atom that may act as a suitable point of attachment for the linker group. It will also be appreciated that the Z moiety may be attached to more than one linker. Thus, the compound of Formula (I) may have more than one linker group, which may be the same or different. In preferred embodiments, the linker groups are the same. In some embodiments, the linker (L) is attached to the wide band gap moiety through a carbon or a nitrogen atom in the wide band gap moiety. In some embodiments, the linker is attached through an oxygen atom. Suitably the linker (L) and wide band gap moiety (Z) are joined by a covalent bond. It will be appreciated that it is beneficial that the electronic properties of the wide band gap material are not substantially changed in the wide band gap moiety. Thus, the electronic properties of the host moiety should be substantially unchanged by the presence of the attached linker. This can be realized by ensuring that there is no substantially discernable additional electronic effect, such as by conjugation, introduced by the linker.

In order to retain the individual electronic properties of the wide band gap moiety and the COT moiety of a compound of Formula (I), preferably neither moiety is subject to additional conjugation due to the presence of the linking group. Moreover, preferably the linking group should not in itself contain any conjugation that will result in substantial alteration of the photophysical or electronic properties of the COT unit with respect to the triplet quenching ability and efficiency.

The skilled person will understand that neither a COT moiety nor a wide band gap moiety of a compound of Formula (I) should be subject to any further conjugation by the linking group and will be readily able to determine suitable non-conjugating linking groups. An example of a non-conjugating linker group is a saturated alkylene chain, for example an alkylene chain with greater than two, or greater than three carbon atoms. In some embodiments, the linker L^{A} is a C₂-C₃₆ or a C₃-C₃₆ alkylene chain, such as C₂-C₂₄, C₂-C₁₆, C₂-C₁₂, C₃-C₃₆, C₃-C₃₀, C₃-C₂₄, C₃-C₁₆, C₃-C₁₂, or C₃-C₁₀, for example n-propylene [—(CH₂)₃—]; n-hexylene [—(CH₂)₆—]; or n-decylene [—(CH₂)₁₀—]. It will be understood that an alkylene chain may be straight chain, or branched. However, the skilled person will understand that examples of a non-conjugating linker group are not so limited. In some embodiments, the linking group may contain one or more additional groups, such as one or more oxygen atoms (e.g., ether or ester linkers), or one or more nitrogen atoms (e.g., amino or amide linkers), or any unsaturated linkers that will not substantially affect the photophysical or electronic properties of the individual wide band gap and COT moieties. The linking group may also be substituted. The location of an oxygen or nitrogen atom in the linker chain should be such that it will not substantially affect the photophysical or electronic properties of the individual wide band gap and COT moieties. In some embodiments, an oxygen/nitrogen atom may be positioned near or adjacent to the COT moiety or wide band gap moiety.

In some embodiments, the linker chain L^{A} may be attached to the Z moiety and/or the COT moiety through an amino, ether, ester or amide linkage, thus forming a linker L^{B}. The linker L^{B} may be attached to the COT moiety and/or the Z moiety through a group X¹ or X², the X¹ group being attached to the COT moiety and the X² being attached to the Z moiety. In some embodiments, X¹ and X² may be individually selected from —O—, —NR¹—, —(CO)O—, —O(CO)—, —(CO)NR²— or —NR²(CO)—. R¹ and R² are each selected from H or alkyl, for example H or C₁₋₁₀ alkyl, especially C₁₋₆alkyl. In some embodiments, one of X¹ or X² may be replaced by a bond. In some embodiments, X¹ and X² are each an oxygen atom, or one of X¹ and X² is oxygen and the other is a bond.

It will be understood that in the context of the present invention, conjugation refers to overlap of three or more p orbitals to provide a n (pi) system resulting in increased electron delocalisation. For example, alternating single and double (or triple) bonds can provide conjugation. Moieties that can participate in conjugation in an organic molecule include n-bonds, e.g. double or triple bonds; heteroatoms with a lone pair of electrons, such as but not limited to O, N or S; radicals such as a carbon atom with a half filled p orbital; and carbocations having a half filled p orbital. It will be appreciated that inclusion of any of these features in a linker wherein the feature is adjacent to or capable of conjugating with the COT or wide band gap host moiety can, subject to steric or conformational restrictions, introduce conjugation and should thus be avoided.

In some embodiments, the COT moiety may be substituted with up to seven substituents, which may each be the same or different. With respect to compounds of Formula (I), n is an integer from 0 to 7. In some embodiments, n is 0. In some embodiments n is 1 or 2. Suitable R substituents will not form conjugation with the COT moiety. Examples of suitable substituents include C₁-C₁₂ alkyl straight chain or branched groups. A further example of an R substituent includes alkoxy groups, such as C₁-C₁₂ alkyoxy straight chain or branched groups. It will be understood that, in order to mitigate against unwanted conjugation, the COT moiety should not bear alkoxy groups on two adjacent ring carbon atoms. In some embodiments, a COT substituent R may be a group -L-Z, wherein L and Z is as hereinbefore defined, thus providing a compound of Formula (If) wherein the COT moiety may be linked to more than one Z moiety wherein each Z moiety, and each linker L, may be the same or different.

As used herein, the term “alkyl” is taken to include straight chain or branched chain monovalent saturated hydrocarbon groups, preferably having greater than two or greater than three carbon atoms, for example 3 to 24 carbon atoms. Examples of a straight chain alkyl group includes propyl, butyl, pentyl, hexyl, heptyl, octyl, decyl, dodecyl, and the like. Examples of a branched chain alkyl group includes isopropyl, isobutyl, sec-butyl, tert-butyl, iso-pentyl, neo-pentyl, and the like.

As used herein, the term “alkylene” refers to bivalent group derived from the removal of a hydrogen atom from two different carbon atoms of an alkyl group and thus may be straight chain or branched. The bivalent alkylene group has two points of attachments to other groups.

As used herein, the term “alkoxy” or “alkoxy group” is taken to include —O— alkyl groups, i.e. alkyl groups bound to an oxygen atom, preferably where the alkyl group has 3 to 20 carbon atoms. The alkoxy group may be straight chain or branched chain alkoxy groups. Examples of a straight chain alkoxy group includes propoxy, butoxy, pentoxy, hexyloxy, heptyloxy, octyloxy, dodecyloxy and the like.

In an exemplary embodiment, the compound of Formula (I), (Ia), (Ib), (Ic) and (Id) is 9,9′-(5-(6-((1Z,3Z,5Z,7Z)-cycloocta-1,3,5,7-tetraen-1-yl)hexyl)-1,3-phenylene)-bis(9H-carbazole), also referred to herein as mCP-C6-COT.

In some other embodiments of the compounds of Formula (I), (Ia), (Ib) or (Ic) include the following exemplary compounds designated Z-C10-COT:

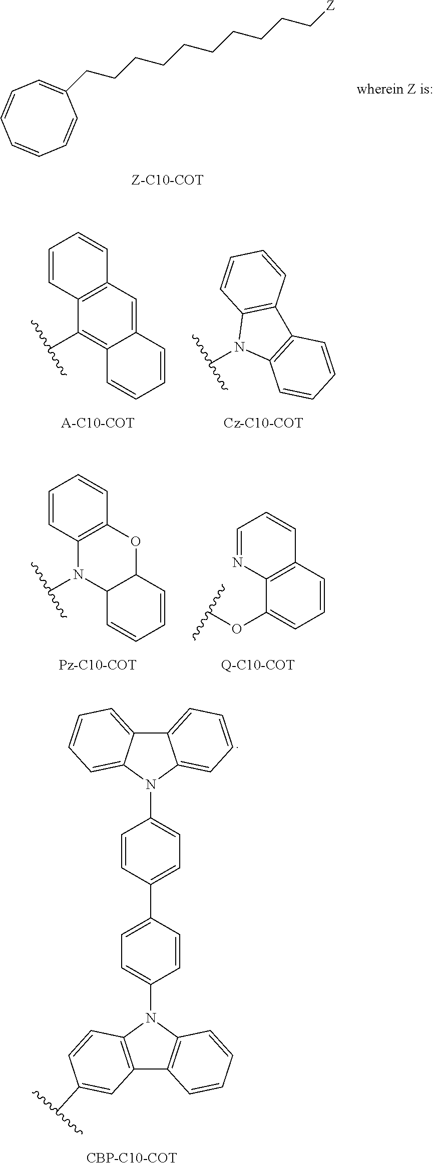

In some embodiments, a compound of Formula (I) or (Ie) is 2,7-bis(2-((1Z,3Z,5Z,7Z)-cycloocta-1,3,5,7-tetraen-1-yl)ethyl)-9,9-dihexyl-9H-fluorene, FI-COT:

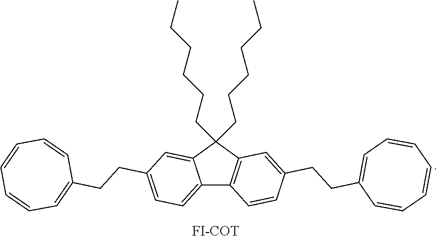

In use, it will be appreciated that the compounds of Formula (I) can be used as part of a mixture of compounds. The two or more compounds may be in any ratio in accordance with the chemical, physical, photophysical or electronic properties required. Those skilled in the art will readily be able to determine the identity and ratios of the compounds depending on the circumstances. In some embodiments, the compounds in such a mixture are selected from compounds of Formula (I).

It will be understood that, unless stated to the contrary, for the purposes of the description below, references to the term “Formula (I)” also applies to the Formulae (Ia), (Ib), (Ic), (Id), (Ie) and (If).

### Compositions of the Invention

The compounds of the invention have good solubility in organic solvents. In some embodiments, the physical and chemical properties, including thermal properties, are not adversely affected by solution processing. In particular, mCP-C6-COT has good solubility properties in common organic solvents, for example chloroform, toluene, or chlorobenzene, and thus is suitable for solution processing.

For use as a TSQ for laser technology, those skilled in the art will understand that a compound of Formula (I) is suitably processed or formulated in accordance with the requirements of its intended use in the absence of any other material such as substrate, binders, plasticisers, polymeric matrices, host matrices, and the like.

The compounds of Formula (I) are soluble in one or more solvents, and thus may be formulated in solution. In some embodiments, the compound may be cast or deposited from solution and the solvent allowed to evaporate to provide a film or coating, such as a thin film. In some embodiments, the compound may be deposited by printing or spraying. In some examples, the compound of Formula (I) is suitably provided as a coating on a substrate. Examples of suitable substrates are well known in the art and will depend on the application. In some embodiments, a substrate is fused silica.

Determination of a suitable solvent (or solvent combination) to form a coating solution comprising a compound of the invention is well within the skill and knowledge in the art. In some examples, the film or coating is deposited from chloroform solution. Methods of coating are well known in the art and may be selected in accordance with the particular application and circumstances. Examples of methods of coating or casting films include, for example, spin-coating, blade coating or hand coating using, for example, a K bar. Other examples include ink-jet printing or spray deposition.

In another aspect, the present invention thus provides a coating or film comprising a compound of Formula (I). In some embodiments, the compound of Formula (I) is mCP-C6-COT.

The required thickness of a coating or film will depend on its intended application. It will be appreciated that the thickness of a coating or film can be controlled by modification of factors during its preparation. In some embodiments, the film thickness can be controlled by altering the speed of rotation during spin coating, or by altering the concentration of the coating solution. Examples of coating solution concentrations include from about 20 mg mL⁻¹ to about 30 mg mL⁻¹ of a compound of Formula (I) in chloroform. In some embodiments, the film or coating is a thin film with a thickness of about 100 nm to about 400 nm; for example from about 120 nm to about 260 nm, or about 140-150 nm. In some examples, a thin film comprising a compound of Formula (I) may be spin coated from a 20 mg mL⁻¹ chloroform solution at 1,500 rpm on a fused silica substrate to obtain film thickness of about 140-150 nm.

In some embodiments, the film or coating is flexible. It will be appreciated that, if required, the coating solution may additionally comprise additives such as plasticisers to improve or modify the physical properties of the film. The selection of any additives will be well within the knowledge of those skilled in the art.

In some preferred embodiments a compound of Formula (I) is suitably formulated with one or more additional components. In some embodiments, the quencher compound of Formula (I) and additional components are combined in a matrix, suitably in a solution, prior to depositing as a film or coating.

When formulated in a composition with an organic semiconductor dye/dopant, the compounds of Formula (I) have been found to exhibit excellent triplet quenching ability under both optical and electrical excitations in the nanosecond (ns) range. In some embodiments, negligible effects on the amplified spontaneous emission (ASE) thresholds is observed. In some embodiments, a complete suppression of singlet-triplet annihilation (STA) is achieved, for example under continuous-wave (CW) excitation. In some embodiments, a 20-fold increase in excited-state photostability of the organic laser dye is achieved under CW excitation.

Accordingly, in a further aspect, the present invention advantageously provides a composition comprising:
a compound of Formula (I) as defined herein; and
an organic semiconductor laser dye.

In some preferred embodiments, a composition of the invention is provided as a coating or film.

Organic semi-conductor laser dyes are well known in the art and are readily available from commercial sources. For example red, green and blue dopants (or dyes) are available from such sources as: Luminescence Technology Corp (Lumtec), Taiwan: (https://www.lumtec.com.tw) and W. Sands Corp (https://www.hwsands.com/category/21.aspx).

Organic semi-conductor laser dyes are also described in for example Jiang, Y. et al. Chem. Soc. Rev. 2020, 49, 5885; Kuehne, A. J. C. & Gather, M. C. Chem. Rev. 2016, 116, 21, 12823; Samuel, I. D. W. &Turnbull, G. A. Chem. Rev. 2007, 107, 1272; and Chenais, S. & Forget, S. Polym. Int. 2012, 61, 390.

In some preferred embodiments, suitable dyes include soluble, solution processable laser dyes. Suitable laser dyes include soluble bis-stilbene dyes. Exemplary dyes include is the blue laser dye BSBCz-EH and the green laser dye BSBCzCN-EH (Mamada, M. et al., Adv. Funct. Mater. 2018, 28, 1802130).

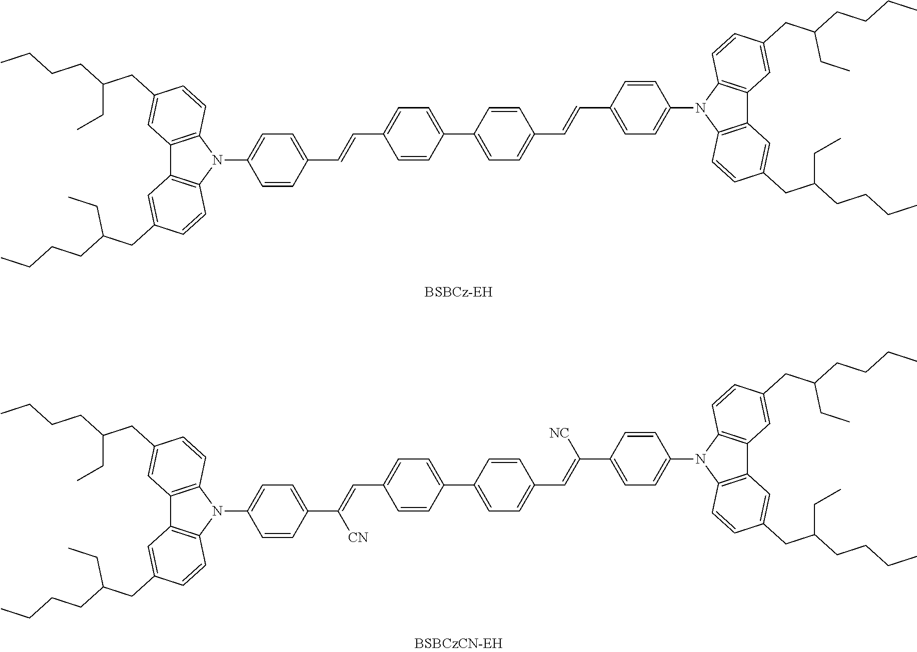

The amount of compound of Formula (I) and the amount of dye present in the composition will depend on the identity of the dye and the compound of Formula (I) in addition to the requirements of the application. Those skilled in the art will be able to determine suitable amounts depending on the circumstances without inventive input.

For example, in some embodiments a compound of Formula (I), may comprise about 1% to about 25% by weight of the composition, for example from about 2% to about 20%; about 2% to about 15%; about 5% to about 20%; about 1% to about 10%; about 1% to about 5%; about 2% to about 8%; about 2.5% to about 10%; or about 3% to about 12% by weight.

The amount of dye/dopant present in a composition of the invention will depend on the identity of the dye, the circumstances and the requirements of the application. Those skilled in the art will be able to determine suitable without inventive input. For example, a dye may comprise about 1% to about 15% by weight of the composition, for example from about 1% to about 10%; about 1% to about 7.5%; about 1% to about 5%; about 1% to about 3%; about 3% to about 7%; about 2.5% to about 5%; about 4% to about 6%; or about 1%, 2%, 3%, 4% or 5% by weight.

### Methods of Synthesis

The compounds of Formula (I) may be prepared from commercially available starting materials and reagents using conventional multistep synthetic routes. The compounds of Formula (I) may be prepared, for example, by analogous routes to those described for specific examples in the reaction schemes below and in the Examples. Suitable reactions are well known and include, for example, nucleophilic aromatic substitution and coupling reactions such as palladium-catalysed Suzuki or Suzuki-Miyaura coupling reactions, and other chemical transformations well known in the art.

The synthesis of mCP-C6-COT is summarised in the scheme below.

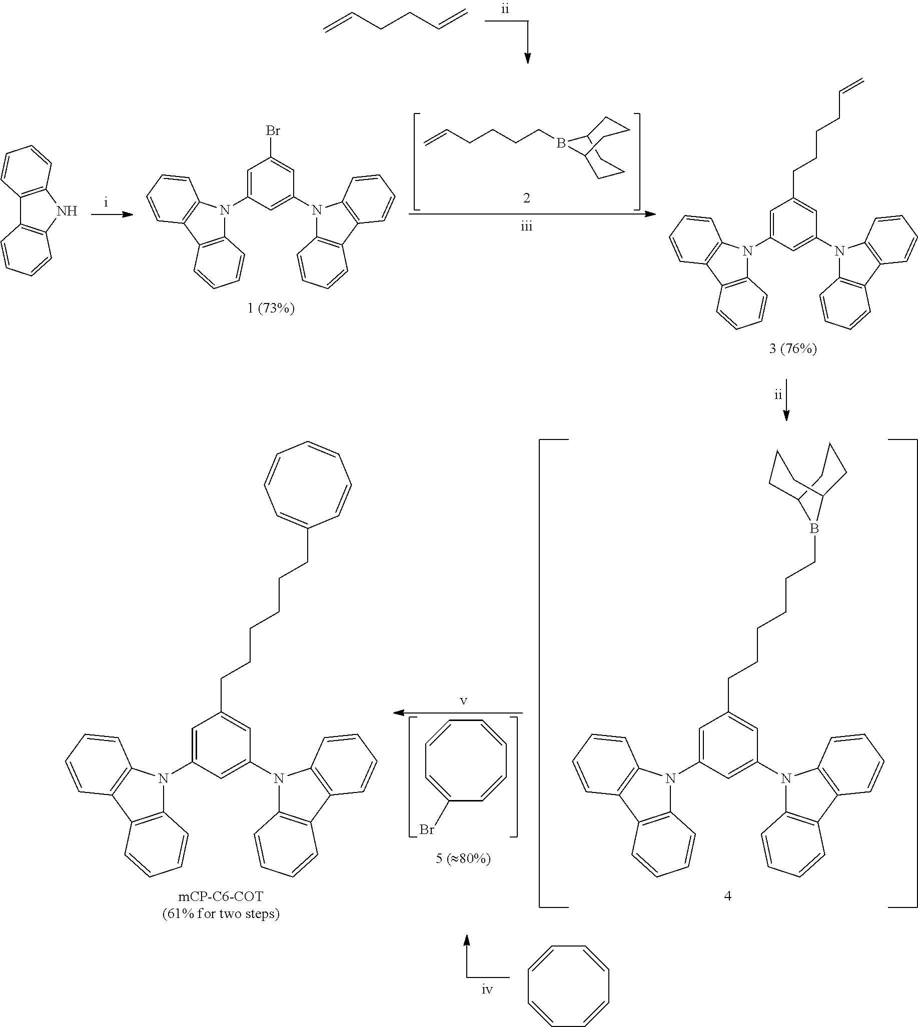

Synthetic route to mCP-C6-COT: ia) ^{t}BuOK, DMSO, Ar_{(g)}, 120° C., 0.5 h, b) 1-bromo-3,5-difluorobenzene, Ar_{(g)}, 140° C., 0.5 h; ii) 0.5 M 9-BBN in THF, Ar_{(g)}, r.t., 2.5-3.5 h; iii) 2, K₂CO₃, Pd(dppf)Cl₂-DCM, DMF, Ar_{(g)}, 60° C., 16 h, 76%; iva) Br₂, DCM, Ar_{(g)}, −70° C., 1 h, b) ^{t}BuOK, THF, Ar_{(g)}, −60° C., 3 h; v) 5, K₂CO₃, Pd(dppf)Cl₂·DCM, DMF, H₂O, Ar_{(g)}, 55-60° C., 17 h.

The synthesis of FI-COT, a compound of Formula (Ie), is summarised below.

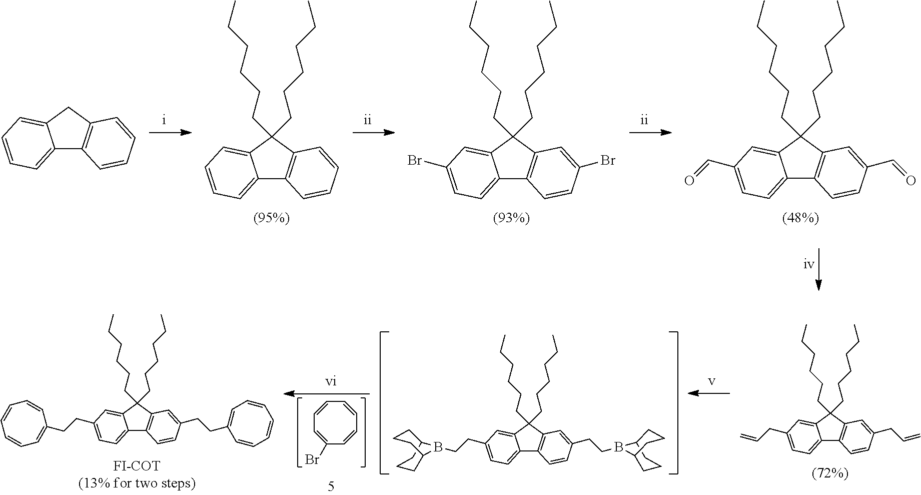

Synthetic route to FI-COT: ia). n-BuLi, THF, −78° C., 1 h, Ar_{(g)}, b) 1-bromohexane, r.t., 16 h, Ar_{(g)}; iia) n-BuLi, THF, −78° C., 0.5 h, Ar_{(g)}, b) DMF, −78° C. to r.t., 16 h, Ar_{(g)}, c) 3 M HCl_{(aq)}, 15 min; iii) 12 (cat.), Br₂, DCM, r.t., 16 h, Ar_{(g)}; iv) MePPh₃I, n-BuLi, THF, 0° C., 15 min, and r.t., 15 h, Ar_{(g)}; v) 9-BBN, THF, 0° C., to r.t., 4 h, Ar_{(g)}; vi) 3.0 M NaOH(aq), Pd(PPh₃)₄, THF, reflux, 19 h, Ar_{(g)}.

It will be understood that these routes may be adapted to prepare other compounds of Formula (I). One skilled in the art would understand that a wide variety of compounds of Formula (I) can be accessed through judicious selection of starting materials, appropriate reagents, various chemical transformations and reaction conditions.

Suitable starting materials and reagents may be available from commercial sources, or may be synthesized using routes well known to those skilled in the art.

For example, bromo-substituted starting materials such as (1) may be prepared using an analogous route to that used in the above reaction scheme using appropriate precursors and reagents. Alternatively, aryl bromides may be prepared from an appropriate precursor in accordance with other well-known methods for the synthesis of aryl bromides, such as a Sandmeyer reaction. Substituted 9-BBN reagents, may be prepared according to well-known methods from the corresponding alkene (e.g. 1-dodecene) and 9-borabicyclo[3.3.1]nonane (9-BBN) (N. Miyamura, et al., J. Am. Chem. Soc. 1989, 111, 314).

It will be appreciated that certain substituents in any of the reaction intermediates or compounds of Formula (I) may be converted to other substituents by conventional methods known to those skilled in the art. Synthetic routes to starting materials and reagents from commercially available synthetic precursors and appropriate chemical transformations are well known in the art and are described in, for example, Richard Larock, Comprehensive Organic Transformations, 2^{nd} Edition, Wiley, ISBN 0-417-19031-4.

It will be appreciated that it may be necessary to protect certain substituents during one or more synthetic procedures. Those skilled in the art will recognize when a protecting group is required. Standard protection and deprotection techniques, such as those described in Peter G. M. Wuts, Greene's Protective Groups in Organic Synthesis, Wiley, New York, 2014 ISBN 9781118057483, may be used. It will be appreciated that protecting groups may be interconverted using conventional means.

The reactions and processes described herein may employ conventional laboratory techniques for heating and cooling, such as thermostatically controlled oil baths or heating blocks and ice baths or solid C02/acetone baths. Use of inert atmospheric conditions such as nitrogen or argon may be employed. Conventional methods of isolation of the desired compound, such as extraction or precipitation techniques, and the like, may be used. Organic solvents or solutions may be dried where required using standard, well-known techniques. Purification of compounds or intermediates may be effected using conventional techniques such as chromatography and/or crystallisation.

### Methods of Use

It has been discovered that the compounds of Formula (I) find application as triplet excited-state quenchers. In particular they are useful in the field of organic laser diodes where they can mitigate against accumulation of triplet excitons which lead to significant losses under continuous wave (CW) or electrical excitation. The compounds of Formula (I) are solution processable, facilitating preparation of coatings and films, such as thin films. In use, compounds of Formula (I) have been shown to have negligible effects on the ASE thresholds. In some embodiments, they have been shown to effect a substantially complete suppression of singlet-triplet annihilation (STA) and a 20-fold increase in excited-state photostability of a dye under CW excitation. When investigated in small-area OLEDs (0.2 mm²) efficient STA suppression was demonstrated by mCP-C6-COT in the nanosecond (ns) range. The solid-state triplet quenchers of compounds of Formula (I) as described herein provide good triplet quenching ability under both optical and electrical excitations in the ns range, coupled with excellent solution processability.

In particular, the compounds of Formula (I) as defined herein find potential utility in organic solid-state lasers; optical communications, (bio-)sensing and opto-electronic applications; laser diodes such as organic semiconductor laser diodes (OSLDs); light-emitting diodes; solar cells; sensors; and photorefractive devices.

These triplet excited-state quenchers finds utility in fields such as data communication and metal-organic plasmonic devices and electrically pumped organic lasers.

In some embodiments, the compounds of Formula (I) are useful in laser technology as triplet excited-state quenchers and may be used in accordance with methods and apparatus well known to those in the art.

Those skilled in the art will appreciate that the invention described herein is susceptible to variations and modifications other than those specifically described. It is to be understood that the invention includes all such variations and modifications which fall within the spirit and scope.

In order that the invention may be readily understood and put into practical effect, particular preferred embodiments will now be described by way of the following non-limiting examples.

### EXAMPLES

General

All commercial reagents and starting materials were used as received unless otherwise stated. Tetrahydrofuran (THF) and N,N-dimethylformamide (DMF) were dried using a vacuum-argon solvent purification system before use. Dimethyl sulfoxide (DMSO) was stirred overnight with calcium hydride (3% w/v), distilled and stored in activated 4 Å molecular sieves under argon. Dichloromethane was dried with calcium hydride (3% w/v) overnight and freshly distilled prior to use.

Material Synthesis and Characterisation

Petroleum with boiling points of 40-60° C. and dichloromethane were distilled prior to use for column chromatography, using Merck LC60A 40-30 silica gel. Solvent ratio used for column chromatography is reported by volume. All 1H spectra were recorded using Bruker Avance 300 or 500 MHz spectrometers in CDCl₃. All chemical shifts (6) were reported in parts per million (ppm) and referenced to the residual solvent peak at b 7.26 ppm in ¹H NMR, and 77.0 ppm for CDCl₃ in ¹³C NMR. Multiplicities were reported as singlet (s), doublet (d), triplet (t), multiplet (m), doublet of doublets (dd) and doublet of triplets (dt); COT-H=cyclooctatetraenyl H, Cz-H=carbazolyl H, Fl-H=fluorenyl H, Q-H=quinolinyl H, Ph-H=phenyl H and V—H=vinyl H. All coupling constants (J) were quoted in Hertz (Hz) and rounded to the nearest 0.5 Hz. Melting point (m.p.) was measured in a glass capillary on a BÜCHI Melting Point B-545 and was uncorrected. Infrared spectra were recorded on a Perkin Elmer Spectrum 1000 FT-IR spectrometer with ATR attachment as solid state. Mass spectra were recorded on a Bruker Esquire HCT (High Capacity 3D ion trap) electrospray ionization (ESI) MS or a BRUKER MicrOTof-Q for the accurate mass in ESI mode. Absorption spectra were recorded on a Varian Cary 5000 UV-Vis-NIR spectrophotometer in 10×10 mm quartz cuvettes and λ_{abs} values are quoted in nm with shoulders denoted as “sh”. Electrochemical studies were conducted using an Epsilon C3 BAS electrochemistry station using glassy carbon working, 0.01 M AgNO₃ in acetonitrile reference, and platinum counter electrodes. All measurements were conducted at room temperature with the sample concentration of 1 mM in dichloromethane (distilled from calcium hydride) and 0.1 M tetra-n-butylammonium perchlorate as the electrolyte. The solution was deoxygenated with argon and a ferricenium/ferrocene (Fc⁺/Fc) couple was used as standard. The scan rate was 100 mV s⁻¹. The thermal gravimetric analysis (TGA) measurement was performed on a Perkin Elmer STA 6000 under 20° C. min-1 under an inert atmosphere, the TGA temperature was quoted at 5% weight loss as the decomposition temperature. Differential scanning calorimetry (DSC) was used to investigate thermal properties with heating and cooling rates of 200° C. min-1 under a nitrogen atmosphere.

Photophysical Measurements

All substrates were cleaned by sonication in acetone and isopropanol, followed by UV-ozone treatment. Thin films were fabricated by spin-coating from a chlorobenzene solution of 1.0 wt % fluorescent dye and mCP-C6-COT at 1,000 rpm for 60 seconds on clean quartz substrates. UV-Vis absorption spectra were measured using Perkin-Elmer Lambda 950-PKA UV-vis spectrophotometer. Photoluminescence (PL) spectra were measured using Horiba Jobin Yvon FluoromMax-4. PLQYs were measured using an absolute PLQY measurement system (Hamamatsu Photonics Quantaurus-QY C11347-01). The measurement error for the obtained QY values on this instrument was ±3%. Transient photoluminescence decay was recorded on a Hamamatsu Photonics Quantaurus-Tau C11367-03. Phosphorescence spectra were recorded on the Hamamatsu Photonics PMA-12 with the LED excitation at 300 nm (Thorlabs M300L4).

Computational Studies

The computations were mainly performed using the computer facilities at the Research Institute for Information Technology, Kyushu University. Molecular orbital calculations were performed using the programs Gaussian 16. The geometries for the ground state were optimized at the B3LYP/6-31+G(d,p) level. The presence of energy minima was confirmed by the absence of imaginary modes (no imaginary frequencies). The time dependent density functional theory (TD-DFT) calculations were conducted at the B3LYP/6-31+G(d,p) level. To numerically achieve accurate values, a fine grid was used. The solvation effect in a solvent was considered by using the polarizable continuum model (PCM). Since the optimization at the excited states for COT and mCP-C6-COT using TD-DFT resulted in convergence failures, TD-B3LYP/6-31+G(d,p)//CIS/6-31+G(d,p) was used, while the optimization at the excited states of mCP was conducted using TD-B3LYP/6-31+G(d,p). The optimized structures for the triplet excited state were also calculated using UB3LYP/6-31+G(d,p).

ASE Measurements

Thin films were prepared by spin-coating from 1.5 wt % chloroform solution at 1,000 rpm for 60 seconds on nonfluorescent glass substrates (MATSUNAMI slide glass S0313). The substrates were cut to use the centre of the substrate with smooth flat surface. ASE properties of the thin films were characterised by optically pumping with a randomly polarised nitrogen gas laser (KEN2020, Usho Optical Systems Co., Ltd.) at an excitation wavelength of 337 nm with a 0.8 ns pulse (operating frequency of 10 Hz). The input laser beam was focused into a stripe with dimensions of 0.6 cm×0.12 cm using a cylindrical lens. Neutral density filters were used to adjust excitation intensity. ASE measurements were performed under a nitrogen atmosphere. Output light emission from the edge of the sample was collected into an optical fiber connected to a spectrometer (Hamamatsu Photonics PMA-12). ASE thresholds were identified from the plot of output versus input intensity

Transient PL Measurements

Thin films were prepared using the same condition as those for photophysical measurements on non-fluorescent glass substrates, which were encapsulated in a glovebox under nitrogen. A CW laser diode (Coherent OBIS LG 355-20) was used to generate excitation light with an excitation wavelength of 355 nm. In these measurements, pulses were delivered using an acousto-optic modulator (Gooch & Housego, MHP085-6DS2) that was triggered with a pulse generator (WF 1974, NF Co.). The excitation light was focused on a 200 μm beam diameter through a lens and slit, and the excitation power was 2.65 mW. The size and power were checked by using a beam profiler (Thorlabs BP209-VIS) and thermal sensors (Ophir Optronics 3A-PF-12 and StarLite). The emitted light intensity was recorded using a photomultiplier tube (PMT) (Hamamatsu Photonics R928, C3830). The PMT response was monitored on a multichannel oscilloscope (Agilent Technologies DS05034A).

Photodegradation Measurements

Thin films were fabricated by spin-coating from 1.2 wt % chloroform solution at 1,000 rpm for 60 seconds on non-fluorescent glass substrates, which were encapsulated in a glovebox. A CW laser diode (NICHIA NDV7375E) was used to generate excitation light with an excitation wavelength of 405 nm. The excitation beam area was 2.5 mm×2.5 mm circle. The excitation power was 200 mW for BSBCz-EH. The emission spectra were recorded using spectrometer (Hamamatsu Photonics PMA-12).

OLED Fabrication

Pre-patterned ITO substrates on a 0.5 mm thick glass were sonicated for 10 min in deionised water followed by 10 min sonication each in acetone and isopropanol. The substrates were dried with nitrogen before exposing them to a 30 min ultraviolet (UV)-ozone cleaning process. A 30 nm layer of PEDOT:PSS was spin coated on cleaned ITO substrate followed by annealing at 120° C. for 20 min. BSBCz-EH and mCP-C6-COT solutions were prepared at a concentration of 7 mg mL⁻¹ in chlorobenzene and stirred for 30 min for thorough mixing. Neat and blend layers were spin coated at 3,000 rpm followed by a 100° C. annealing for 10 min to dry out the solvent. TPBi, LiF and Al were evaporated in one go with the help of masks at a pressure below 10-6 Torr. Devices were encapsulated and UV treated to avoid degradation in air.

Transient EL Measurements

Pulse measurements were done with AVTECH Electrosystems Ltd. pulse generator, AV1011B1-B, having a range of 100 ns to 1 ms. The maximum voltage achieved can be 100 V with an ultra-fast rise and fall time of 2 ns. A calibrated photomultiplier tube (PMT) was used to collect EL data (Hamamatsu H10721-20) having a 0.57 ns response time. A high speed current probe UHF711 from Integrated Sensor Technology was used to measure current with a rated response of less than 0.5 ns. Teledyne LeCroy digital storage oscilloscope (Wavesurfer 900 series), 2 GHz and 10 Gs s⁻¹ was used to record pulse data. Further details can be obtained from literature [Ahmad, V. et al. Adv. Opt. Mater. 2018, 6, 1800768].

Transient Absorption Spectroscopy (TAS) Measurements

Nano-second TAS for mCP and mCP-C6-COT were performed in acetonitrile solutions using a broadband pump-probe spectrometer (EOS, Ultrafast Systems, LLC). An Amplified laser system (spitfire ACE, spectra physics) delivering ca. 100 fs laser pulses at 800 nm with a repetition rate of 1 kHz was the excitation source. The laser pulses were coupled to an OPA system (Topas Prime, Light Conversion) to generate “pump” pulses tuned at 330 nm. The samples were prepared by dissolving mCP/mCP-C6-COT in acetonitrile to achieve an optical density of 0.4 at 330 nm in quartz cuvette with the optical path length of 2 mm. A white light continuum ‘probe’ (ca. 380-900 nm) was generated using a pulsed Nd:YAG based Leukos-STM super continuum light source. The timing of the ‘probe’ pulses was controlled electronically via the sync trigger from the amplified laser system. The sample solutions were stirred continuously to avoid any degradation during the measurements; absorption spectra were measured before and after the measurements to confirm no degradation due to the pump beam.

### Example 1: Preparation of mCP-C6-COT

### Preparation of 9,9′-(5-bromo-1,3-phenylene)bis(9H-carbazole), 1

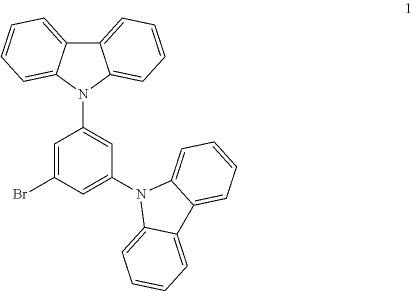 [SMILES: Brc1cc(-n2c3ccccc3c3ccccc32)cc(-n2c3ccccc3c3ccccc32)c1]

A mixture of 9H-carbazole (1.64 g, 9.84 mmol) and potassium tertbutoxide (1.30 g, 11.6 mmol) was deoxygenated using vacuum and back-filled with Ar_{(g)}. This was repeated three times. Anhydrous DMSO (stirred overnight with 3 wt % CaH₂, distilled and stored in 4 Å molecular sieves, 5.0 mL) was added to the mixture and the solution was stirred in an oil bath heated at 120° C. under Ar_{(g)} for 30 minutes, during which the yellow solution turned into orange. 1-Bromo-3,5-difluorobenzene (0.60 mL, 5.21 mmol) was added to the mixture and the reaction was stirred in an oil bath heated at 140° C. under Ar_{(g)} for 2 hours, during which the solution turned into grey with white precipitations. The reaction was cooled to room temperature. Dichloromethane (70 mL) and water (100 mL) were added and the two layers were separated. The aqueous layer was extracted with dichloromethane (2×30 mL). All organic layers were combined, washed with water (2×150 mL), brine (150 mL), dried over anhydrous magnesium sulfate and filtered. The filtrate was collected and the solvent removed under reduced pressure to reveal an off-white solid. The solid was purified by column chromatography over silica using dichloromethane/light petroleum (1:8) as eluent to give 1 as a white solid (1.76 g, 73%). ¹H NMR (500 MHz, CDCl₃) δ 7.31-7.35 (4H, m, Cz-H), 7.44-7.48 (4H, m, Cz-H), 7.55 (4H, d, J=8.0, Cz-H), 7.80 (1H, t, J=2.0, Ph-H), 7.87 (2H, d, J=2.0, Ph-H), 8.15 (4H, d, J=7.5, Cz-H); ¹³C NMR (125 MHz, CDCl₃) δ 109.6, 120.6, 120.8, 123.8, 123.9, 124.1, 126.4, 128.7, 140.3, 140.5.

### Preparation of 9,9′-(5-(hex-5-en-1-yl)-1,3-phenylene)bis(9H-carbazole), 3

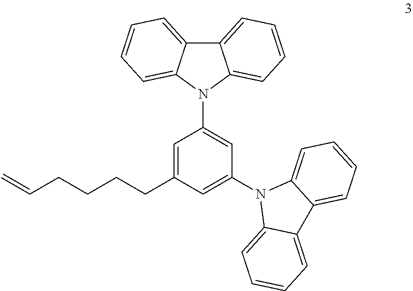 [SMILES: C=CCCCCc1cc(-n2c3ccccc3c3ccccc32)cc(-n2c3ccccc3c3ccccc32)c1]

9-Borabicyclo[3.3.1]nonane (0.5 M in THF, 20 mL, 10.0 mmol) was added dropwise to a solution of 1,5-hexadiene (12 mL, 98.7 mmol) under Ar_{(g)} at room temperature. The mixture was stirred at room temperature for 2.5 hours. The excess 1,5-hexadiene and solvent were removed under reduced pressure to give a cloudy slightly viscous liquid. A mixture of the borane from above, 1 (1.45 g, 2.98 mmol), K₂CO₃ (2.05 g, 14.8 mmol) and Pd(dppf)Cl₂—CH₂Cl₂ (93.0 mg, 127 μmol) was deoxygenated using vacuum and back-filled with Ar_{(g)}. This was repeated three times. Anhydrous DMF (14 mL) was added to the mixture and the solution was stirred in an oil bath heated at 60° C. under Ar_{(g)} for 16 hours. The reaction was cooled to room temperature. Diethyl ether (80 mL) and water (150 mL) were added to the mixture. The two layers were separated. The aqueous layer was extracted with diethyl ether (2×80 mL). All organic layers were combined, washed with water (2×150 mL), brine (120 mL), dried over anhydrous magnesium sulfate and filtered. The filtrate was collected and the solvent removed under reduced pressure to reveal a brown-orange gum. The crude was purified by column chromatography over silica using dichloromethane/light petroleum (1:7) as eluent to give 3 as a white solid (1.11 g, 76%); m.p.: 114.5-116.0° C.; vₘₐₓ(solid)/cm⁻¹: 706, 747, 1227, 1334, 1443, 1462, 1592, 2849, 2931, 3054; λₘₐₓ (dichloromethane)/nm: 242 (log ε/dm³ mol⁻¹ cm⁻¹ 4.98), 260 sh (4.54), 283 sh (4.57), 293 (4.43), 327 (3.96), 340 (4.03). ¹H NMR (500 MHz, CDCl₃) δ 1.59-1.65 (2H, m, CH₂), 1.83-1.90 (2H, m, CH₂), 2.18-2.23 (2H, m, CH₂), 2.90 (2H, t, J=7.5, Ph-CH₂), 5.03-5.13 (2H, m, V—H), 5.86-5.94 (1H, m, V—H), 7.34-7.38 (4H, m, Cz-H), 7.48-7.52 (4H, m, Cz-H), 7.57 (2H, d, J=2.0, Ph-H), 7.61 (4H, d, J=8.5, Cz-H), 7.71 (1H, t, J=2.0, Ph-H), 8.21 (4H, d, J=8.0, Cz-H); ¹³C NMR (125 MHz, CDCl₃) δ 28.4, 30.6, 33.5, 35.7, 109.7, 114.7, 120.2, 120.4, 122.5, 123.5, 125.8, 126.1, 138.5, 139.1, 140.6, 146.4; m/z (ESI): calculated for C₃₆H₃₀N₂ [M]: 490.2 (100%), 491.2 (39%), 492.3 (7%); found C₃₆H₃₀N₂ [M]: 490.7 (100%), 491.6 (53%), 492.6 (20%).

### Preparation of (1E,3Z,5Z,7Z)-1-bromocycloocta-1,3,5,7-tetraene, 5

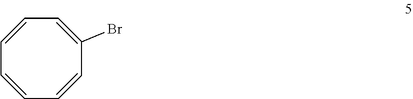

A solution of cyclooctatetraene (8.4 mL, 74.6 mmol) in anhydrous dichloromethane (50 mL) was cooled to −70° C. under argon. A solution of bromine (3.8 mL, 74.2 mmol) in anhydrous dichloromethane (40 mL) was slowly added over 10 minutes into the cold solution of cyclooctatetraene. The solution was stirred at −70° C. under argon for 1 hour to give a pale-yellow solution. A suspension of potassium tert-butoxide (11.2 g, 100 mmol) in anhydrous tetrahydrofuran (40 mL) was added to the cold yellow solution of cyclooctatetraene. The mixture was then stirred at −60° C. under argon for 3 hours and poured into iced water (200 mL). The mixture was extracted with diethyl ether (3×250 mL), washed with water (3×400 mL), dried over anhydrous magnesium sulfate and filtered. The filtrate was collected and the solvent removed under reduced pressure to reveal an amber yellowish oil. ¹H NMR suggested that the crude mixture contained both the desired product (80%) and starting material cyclooctatetraene (20%). The mixture was used for next step without further purification.

### Preparation of 9,9′-(5-(6-((1Z,3Z,5Z,7Z)-cycloocta-1,3,5,7-tetraen-1-yl)hexyl)-1,3-phenylene)bis(9H-carbazole), mCP-C6-COT

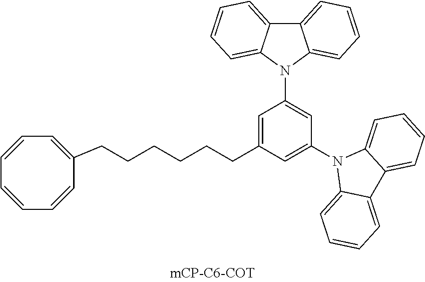 [SMILES: C1=C\C=C/C(CCCCCCc2cc(-n3c4ccccc4c4ccccc43)cc(-n3c4ccccc4c4ccccc43)c2)=C\C=C/1]

A solution of [3] (803 mg, 1.64 mmol) in anhydrous tetrahydrofuran (2.5 mL) was cooled to 0° C. under argon. 9-Borabicyclo[3.3.1]nonane (0.5 M in tetrahydrofuran, 5.0 mL, 2.50 mmol) was added dropwise to the cold solution. The resulting light yellow mixture was stirred at room temperature for 3.5 hours. DMF/H₂O (5.0 mL, 4:1 v/v, bubbled with nitrogen gas for 3 h), [5] (0.7 mL, 5.43 mmol), K₂CO₃ (894 mg, 6.65 mmol) and Pd(dppf)Cl₂·CH2Cl2 (72.1 mg, 100 μmol) were added to the mixture. The reaction was then deoxygenated using vacuum and back-filled with Ar_{(g)}. This was repeated three times. The solution was stirred in an oil bath heated at 55° C. under Ar_{(g)} for 17 hours. The resulted brown-reddish solution was cooled to room temperature and volatile solvent was removed under reduced pressure. The mixture was diluted with ethyl acetate (150 mL) and washed with water (100 mL). The aqueous layer was extracted with ethyl acetate (100 mL). All organic layers were combined, washed with water (5×100 mL), brine (50 mL), dried over anhydrous magnesium sulfate and filtered. The filtrate was collected and the solvent removed in vacuo to reveal a viscous brown-yellowish oil. The crude was purified by column chromatography over silica using petroleum as eluent to give mCP-C6-COT as a colourless solid (594 mg, 61%); m.p.: 71.9-73.1° C.; Td(5%)=408° C.; vₘₐₓ(solid)/cm⁻¹: 704, 722, 745, 1227, 1311, 1332, 1442, 1461, 1591, 2853, 2926, 2998, 3049; Amax (dichloromethane)/nm: 241 (log ε/dm³ mol⁻¹ cm⁻¹ 4.93), 289 sh (4.53), 293 (4.60), 327 (3.92), 340 (3.98). ¹H NMR (500 MHz, CDCl₃) δ 1.40-1.49 (6H, m, CH₂), 1.75-1.82 (2H, m, CH₂), 2.03-2.07 (2H, m, —CH₂-COT), 2.84 (2H, t, J=8.0, Ph-CH₂), 5.54 (1H, br s, COT-H), 5.70-5.80 (6H, m, COT-H), 7.30-7.34 (4H, m, Cz-H), 7.43-7.47 (4H, m, Cz-H), 7.50-7.56 (6H, m, Ph-H & Cz-H), 7.64 (1H, t, J=2.0, Ph-H), 8.16 (4H, d, J=7.5, Cz-H); ¹³C NMR (125 MHz, CDCl₃) δ 28.3, 28.8, 29.1, 31.2, 35.8, 37.6, 109.7, 120.2, 120.4, 122.4, 123.5, 125.8, 126.07, 126.14, 130.8, 131.1, 131.9, 132.4, 134.4, 139.0, 140.6, 144.5, 146.7; m/z (ESI): calculated for C₄₄H₃₈N₂ [M]: 594.3 (100%), 595.3 (48%), 596.3 (11%); found C₄₄H₃₈N₂ [M]: 594.4 (100%), 595.4 (53%), 596.4 (28%); C₄₄H₃₈N₂ requires C, 88.9; H, 6.4; N, 4.7%. found: C, 88.9; H, 6.4; N, 4.7%.

### Example 2: Preparation of FL-COT

### Preparation of 9,9-dihexyl-2,7-divinyl-9H-fluorene

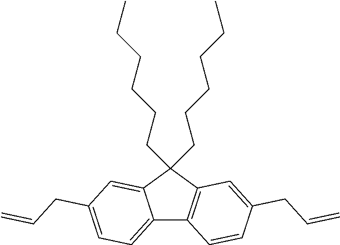 [SMILES: C=CCc1ccc2c(c1)C(CCCCCC)(CCCCCC)c1cc(CC=C)ccc1-2]

9,9-Dihexyl-2,7-divinyl-9H-fluorene was prepared according to the method described in Adkins, C. T. & Harth, E., Macromolecules 2008, 41, 3472. The desired 9,9-dihexyl-2,7-divinyl-9H-fluorene was obtained as a colourless oil (671 mg, 72%). ¹H NMR (300 MHz, CDCl₃) δ 0.60-0.70 (4H, m, CH₂), 0.76 (6H, t, J=6.5, CH₃), 0.90-1.20 (12H, m, CH₂), 1.94-1.99 (4H, m, Fl-CH₂), 5.26 (2H, d, J=11.0, V—H), 5.80 (2H, dd, J=17.5 & 0.5, V—H—), 6.81 (2H, dd, J=17.5 & 11.0, V—H), 7.36-7.41 (4H, m, Fl-H), 7.62 (2H, d, J=8.0, Fl-H); ¹³C NMR (75 MHz, CDCl₃) δ 14.0, 22.6, 23.7, 29.7, 31.4, 40.4, 54.9, 113.0, 119.7, 120.5, 125.2, 136.4, 137.4, 140.7, 151.3.

### Preparation of 2,7-bis(2-((1Z,3Z,5Z,7Z)-cycloocta-1,3,5,7-tetraen-1-yl)ethyl)-9,9-dihexyl-9H-fluorene, FI-COT

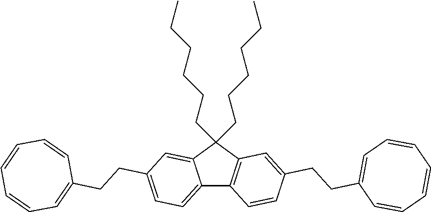 [SMILES: CCCCCCC1(CCCCCC)c2cc(CCC3=C/C=C\C=C/C=C\3)ccc2-c2ccc(CCC3=C/C=C\C=C/C=C\3)cc21]

A solution of 9,9-dihexyl-2,7-divinyl-9H-fluorene (537 mg, 1.38 mmol) in anhydrous tetrahydrofuran (2 mL) was cooled to 0-2° C. under argon. 9-Borabicyclo[3.3.1]nonane (0.5M in tetrahydrofuran, 6.0 mL, 3.00 mmol) was added dropwise to the cold solution. The resulting light yellow mixture was stirred at room temperature for 4 hours to give the borane product. 3M aqueous sodium hydroxide (3.5 mL, 10.5 mmol), tetrakis(triphenylphosphine)palladium(0) (177 mg, 0.153 mmol) and 5 (0.56 mL, 4.36 mmol) were added under argon into the solution of the borane product. The mixture was heated at reflux in an oil bath set at 90° C. under argon protection for 19 hours. The resulting blackish solution was cooled to room temperature and solvent was removed under reduced pressure. The mixture was diluted with 1:1 (v/v) mixture of ethylacetate:petroleum spirit (100 mL), washed with brine (100 mL), dried over anhydrous sodium sulfate, and filtered. The filtrate was collected and solvent removed under reduced pressure to give a yellow solid. The crude was purified by column chromatography over silica using petroleum as eluent to give FI-COT (104 mg, 13%). Found: C, 90.5; H, 9.3. C₄₅H₅₄ requires C, 90.9; H, 9.2%. ¹H NMR (500 MHz, CDCl₃) δ 0.58-0.62 (4H, m, CH₂), 0.76 (6H, t, J=7.0, CH₃), 0.88-1.12 (12H, m, CH₂), 1.90-1.93 (4H, m, Fl-CH₂), 2.37 (4H, t, J=8.0, COT-CH₂), 2.79 (4H, t, J=7.5, CH₂—CH₂-COT), 5.53 (2H, s, COT-H), 5.75-5.85 (12H, m, COT-H), 7.12 (2H, dd, J=7.5 & 1.0, Fl-H), 7.16 (2H, s, Fl-H), 7.54 (2H, d, J=7.5, Fl-H). ¹³C NMR (125 MHz, CDCl₃) δ 14.0, 22.6, 23.7, 29.7, 31.5, 35.4, 40.0, 40.5, 54.7, 119.0, 122.9, 126.8, 127.0, 130.9, 131.4, 132.0, 132.0, 132.4, 134.4, 139.0, 140.4, 143.9, 150.8; m/z (ESI: DCM/MeOH): calculated for C₄₅H₅₄Na [M+Na]⁺: 617.4123; found: 617.1368.

Thermal and Electrochemical Properties

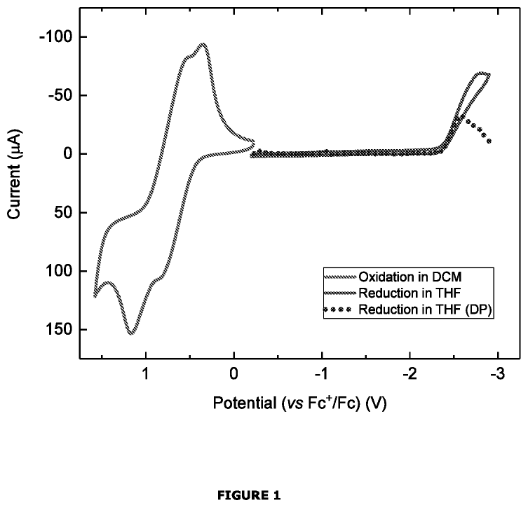

Thermal properties of mCP-C6-COT were studied by using thermogravimetric analysis (TGA) and differential scanning calorimetry (DSC). High thermal stability with a 5% weight loss temperature at 408° C. was found, which is much higher than that (280° C.) of mCP (J. Mater. Chem. 2012, 22, 16114-16120). DSC showed that mCP-C6-COT has a glass transition temperature (Tg) at 55° C., which is comparable to that (60° C.) of mCP. In contrast to the liquid form of COT at ambient conditions, mCP-C6-COT is in a solid state with a melting point (Tₘ) of 73° C. Coupled with the high thermal properties, mCP-C6-COT is solid at room temperature, making it useful for thin-film devices and applications. The electrochemical properties of mCP-C6-COT were investigated by using electrochemistry to show redox behaviours arising from the COT and carbazole species, respectively, which can be attributed to the non-conjugated linkage of the two electroactive species with different electronic properties (see FIG. 1). The oxidations are believed to correspond to the carbazolyl units of mCP-C6-COT, the reduction can be attributed to the COT moiety.

Photophysical Properties and TD-DFT Calculations

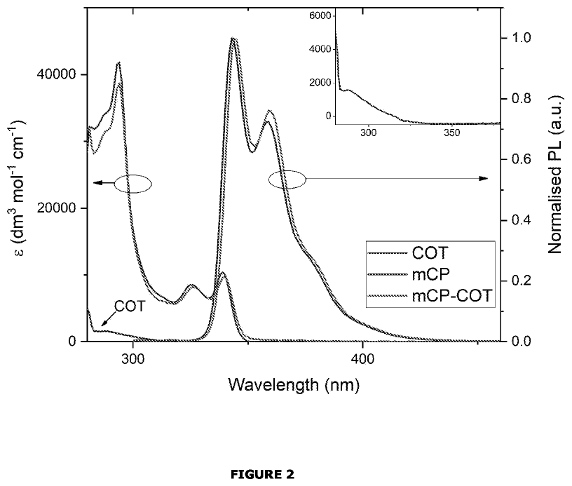

Photophysical properties of mCP-C6-COT were first investigated in toluene and compared to mCP and COT. The steady-state solution absorption and photoluminescence (PL) spectra and the absorption spectrum of COT, mCP-C6-COT, and mCP were co-plotted in FIG. 2.

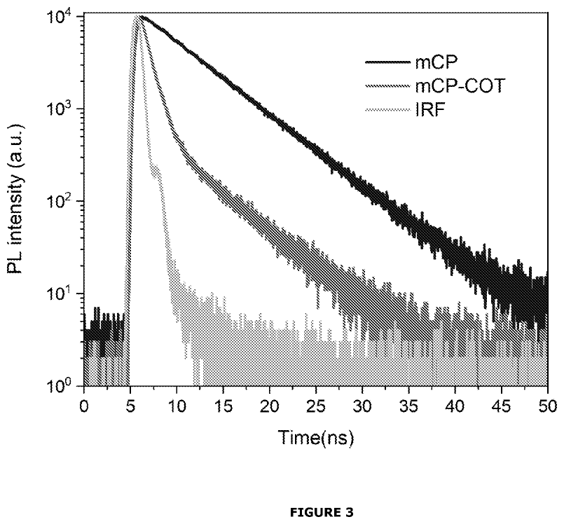

As can be seen from FIG. 2, the solution absorption spectrum of mCP-C6-COT is essentially the same with mCP, suggesting the transition is predominated by the mCP moiety whereas the small red-shifts are attributed to non-conjugated linkage. Coupled with the fact that COT has low molar extinction coefficient, the low energy absorption of mCP-C6-COT peaked at 339 nm can be assigned as n→n* transition from the mCP moiety. While no PL was observed for COT, mCP-C6-COT showed weak PL with a solution photoluminescence quantum yield (PLQY) of 6±3% (in toluene). Thus, mCP-C6-COT shares a similar PL spectrum to mCP with PL peaks at 344 and 343 nm, respectively, indicating the similar emission species due to the non-conjugation of the emissive mCP and non-emissive COT in the molecule. The considerable reduction in solution PLQY of mCP-C6-COT than that (43±3%) of mCP can be attributed to the quenching by the COT moiety attached since it has lower singlet and triplet energies than the mCP unit. Such PL quenching is consistent with a much shorter excited-state lifetime of 1.5 ns for mCP-C6-COT, as determined by time-correlated single-photon counting (TCSPC) with a 3^{rd} order fitting (see FIG. 3) compared to that (5.3 ns in toluene) of mCP. Moreover, the phosphorescence of mCP-C6-COT was significantly reduced, compared to mCP from low-temperature PL measurements, indicating efficient triplet quenching by the COT component.

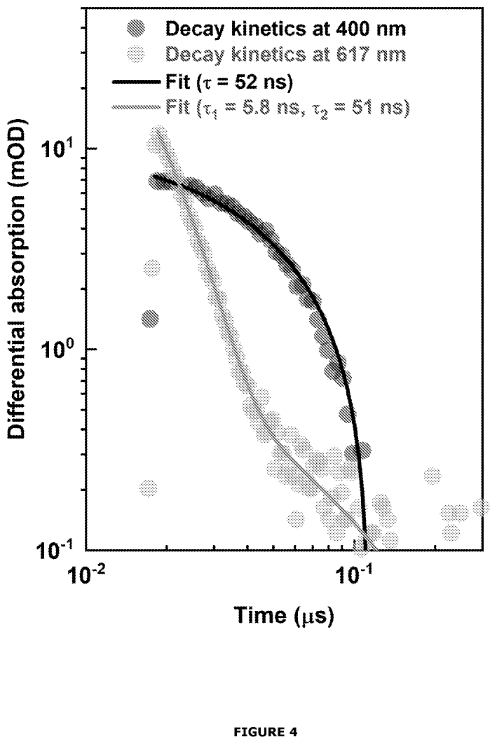

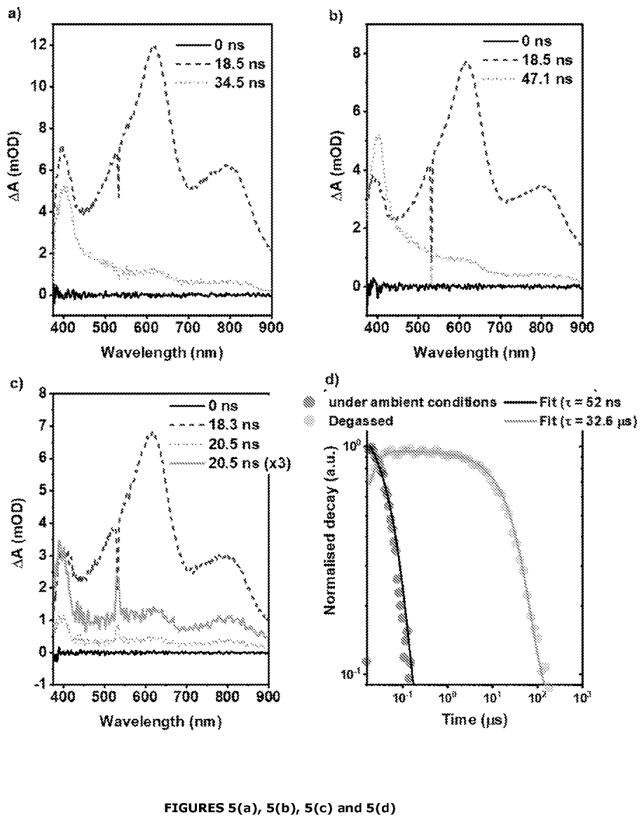

In order to gain further evidence of triplet quenching, nanosecond transient absorption spectroscopy (TAS) was performed for mCP and mCP-C6-COT in acetonitrile. In ambient conditions, mCP showed long-lived excited-state absorption band with maximum at 400 nm (decay lifetime of 52 ns) and broad short lived excited-state feature with maximum at 617 nm (bi-exponential lifetime of 5.8 and 51 ns) (FIGS. 4 and 5). Herein, the decay kinetics of the short-lived absorption band (5.8 ns) was found to match closely with the singlet emission decay obtained from TCSPC measurements (5.3 ns) suggesting this transient absorption band arises due to the singlet excited-state absorption. In order to obtain further insights into the long-lived feature (τ≈50 ns) TAS was performed for mCP under deoxygenated conditions. For deoxygenated solution (degassed using a freeze-pump-thaw method), the lifetime of the long-lived feature increased by more than two orders of magnitude (τ≈32 μs), suggesting this transient absorption band arises due to the triplet excited-states that were otherwise quenched by molecular oxygen under ambient conditions. In case of a deoxygenated mCP-C6-COT solution, similar singlet and triplet excited state absorption bands were observed. The decay lifetime of singlet excited-state absorption band was found to be 0.9 and 7.3 ns which is similar to the singlet emission lifetime obtained in the TCSPC measurements. Furthermore, triplet excited-state absorption decay of the mCP moiety at 400 nm was found to be significantly quenched (τ≈26 ns). The extremely shortened decay lifetime of mCP's triplet excited-state absorption in mCP-C6-COT suggests ultra-fast transfer of triplet excitons from the mCP unit to the COT moiety.

Theoretical calculation showed that mCP-C6-COT has two triplet energy levels, behaving like a non-vertical triplet quencher of COT. At ground state, the optimised structure of the COT moiety in mCP-C6-COT was a non-planar tub-shaped conformation. The vertical excited-state energies for the first singlet (S₁-ver, 3.88 eV) and triplet (T₁-ver, 3.15 eV) of mCP are higher than those (3.27 and 2.22 eV, respectively) of COT. Hence, the S₂-ver (3.89 eV) of mCP-C6-COT corresponds to the S₁-ver of mCP, and the S₁-ver (3.32 eV) and T₁-ver (2.28 eV) of mCP-C6-COT were nearly the same as those of COT, where the S₂-ver and S₁-ver transitions of mCP-C6-COT are mainly populated over mCP moiety [HOMO→LUMO+1 (57%)] and COT moiety [HOMO-2→LUMO (100%)], respectively. However, the S₁-ver of COT is forbidden (with an oscillator strength of 0), which agrees with its absorption spectrum, appearing at shorter wavelength than mCP (FIG. 2). On the other hand, the S₁-ver of mCP-C6-COT appears to be an allowed transition because of the slight spreading of its MOs over the alkyl linker, which might be related to the decrease in PLQY for the doped films. The structure relaxation with planarisation in the excited states resulted in a significant decrease of the energies (see S₁-adi and T₁-adi for the adiabatic excitation), and the triplet quenching by COT is known to include non-vertical triplet energy transfer with conformational changes. Although the S₁-adi and T₁-adi energies were slightly different for COT and mCP-C6-COT, it is considered that mCP-C6-COT can also effectively quench triplet excitons with the same mechanism because of its low T₁-adi energy.

When compared to mCP, mCP-C6-COT has excellent solubility in common organic solvents, thus allowing for good-quality thin-film formation from solution processing. This is desirable for low-cost room-temperature device fabrication using methods such as spin-coating or ink-jet printing.

A solution-processable version of the state-of-the-art organic semiconductor BSBCz dye (Aimono, T. et al. Appl. Phys. Lett. 2005, 86, 071110; Sandanayaka, A. S. D. et al. Sci. Adv. 2017, 3, e1602570) was selected. Thus, BSBCz-EH was selected as an active organic semiconductor laser dye for mCP-C6-COT triplet quenching studies (Mamada, M. et al., Adv. Funct. Mater. 2018, 28, 1802130).

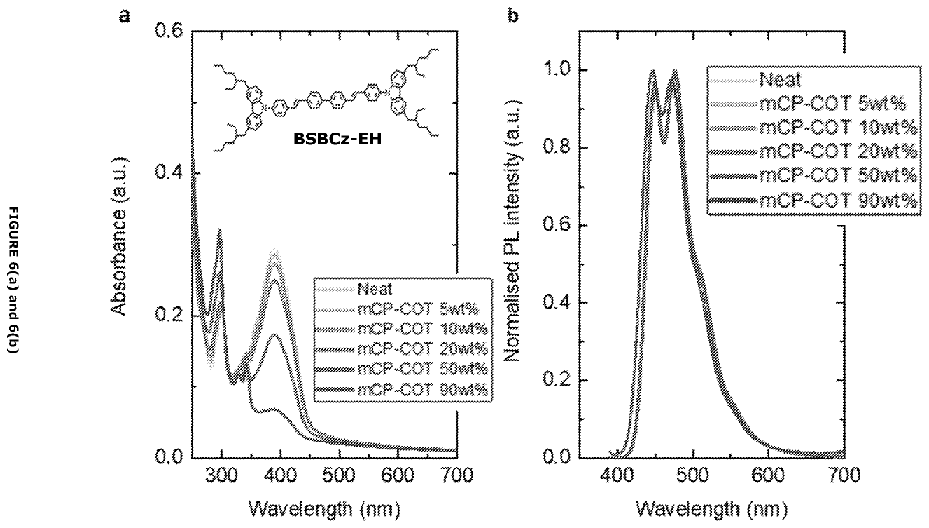

Steady-state neat and blend-film absorption and PL spectra of BSBCz-EH with various mCP-C6-COT blend concentrations (i.e., 1 wt %, 3 wt %, 5 wt %, 10 wt %, 20 wt %, 50 wt % and 90 wt %) are shown in FIG. 6. The absorption and PL maxima are nearly constant, suggesting no particular interactions in the excited states of BSBCz-EH.

Triplet Quenching Studies Under Optical Excitation

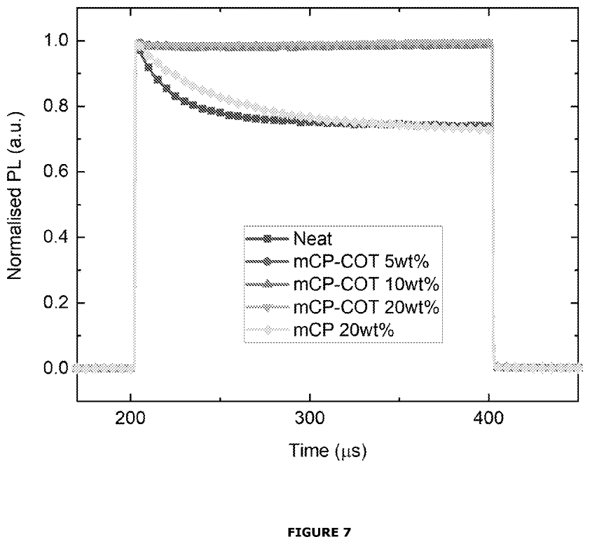

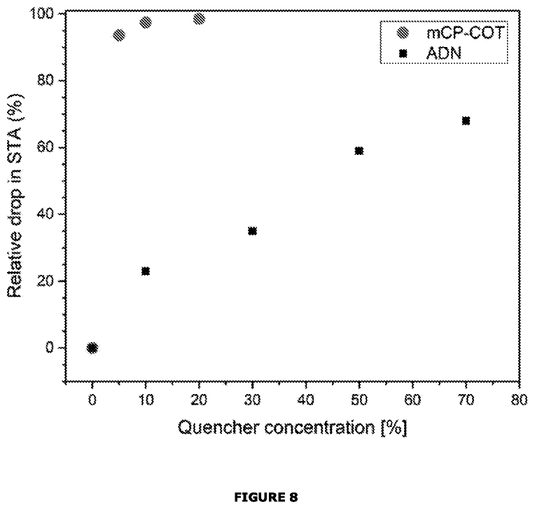

PL transient responses of encapsulated BSBCz-EH neat and blend films with different mCP-C6-COT blend concentrations (5 wt %, 10 wt %, and 20 wt %) as well as a blend film with 20 wt % mCP were investigated. These spin-coated thin films were excited at 355 nm using a circular beam with a diameter of 200 μm. The excitation power, pulse width and pulse interval of the laser beam were 2.65 mW, 200 μs, and 10 ms, respectively. All obtained PL spectra were normalised at their initial PL intensities as shown in FIG. 7. A significant decrease in transient PL intensity under CW excitation for BSBCz-EH neat and the blend films (with 20 wt % mCP) can be clearly observed. The initial PL intensities of both films were reduced by approximately 25% under a pulse width of 200 μs. These reductions can be attributed to quenching of the emissive singlet excitons [i.e., singlet-triplet annihilation (STA)] due to accumulation of the long-lived triplet excited-state generated via intersystem crossing. In contrast, almost completely no reduction in the PL intensity for the blend films of mCP-C6-COT was observed, regardless of blend concentrations, indicating absolute suppression of STA, which in turns advocates the advantageous triplet quenching ability of mCP-C6-COT. A comparative analysis of the triplet quenching performance of ADN and mCP-C6-COT was conducted to show the relative drop in the initial STA in these two systems (FIG. 8). These results support the superior triplet management properties of mCP-C6-COT, since at the 10 wt % concentration mCP-C6-COT removes over 98% STA present originally in the neat system, while the same concentration of ADN results in approximately 25% reduction of STA (FIG. 8).

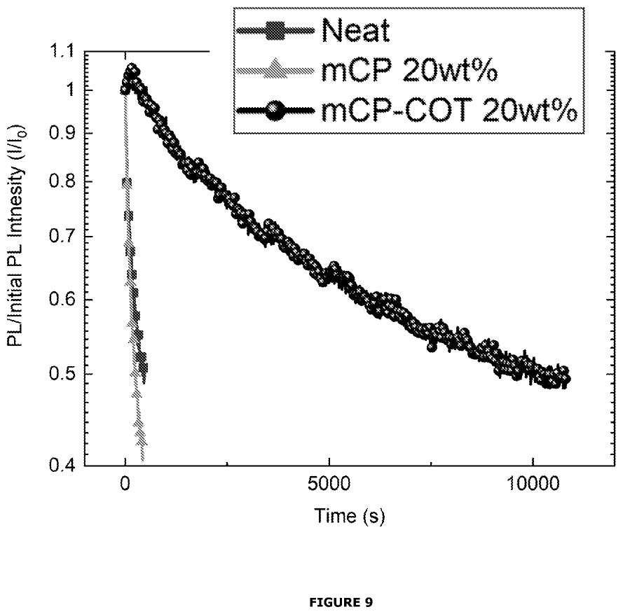

Given that triplet excited-states have been known to photodegrade BSBCz dyes, the effect of mCP-C6-COT on the photostability of BSBCz-EH was investigated. An encapsulated BSBCz-EH neat film, a blend film with 20 wt % mCP, and a blend film with 20 wt % mCP-C6-COT were excited under CW photoexcitation with a power of 200 mW cm⁻² at 405 nm. The PL intensity/initial PL intensity (I/I₀) curves of these films are shown in FIG. 9. The times required for the corresponding films to reach half of their initial PL intensity (i.e., I/I₀=0.5) were found to be 428, 252, and 10,000 s, respectively. This shows that a 20 wt % additive of mCP-C6-COT resulted in more than 20-fold increase in the sustenance of the PL duration, compared to the neat films, indicating the advantageous triplet quenching ability of mCP-C6-COT.

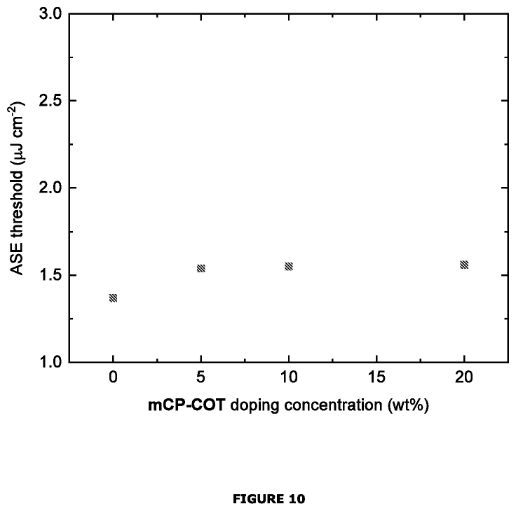

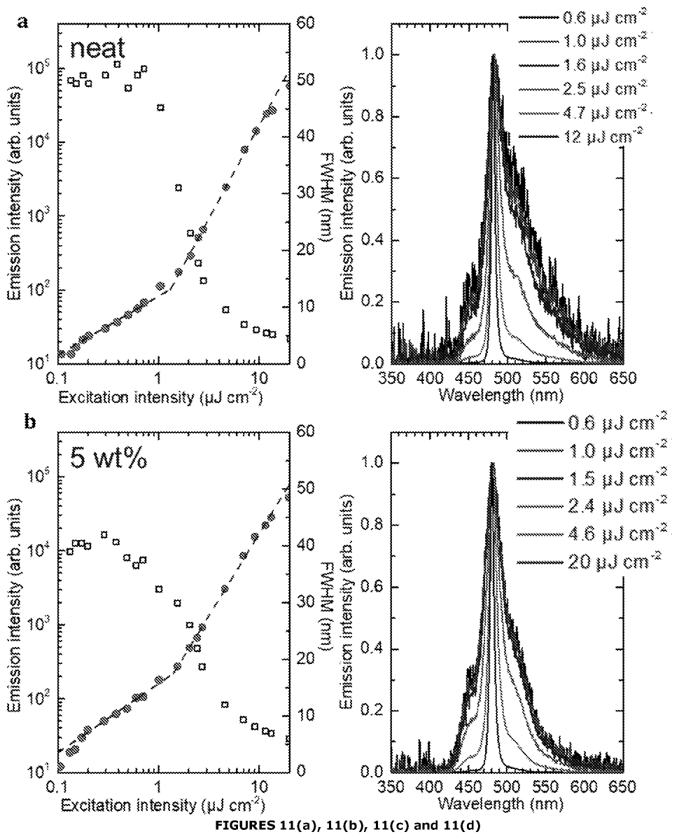

ASE thresholds of neat and blend films of BSBCz-EH with mCP-C6-COT at 5 wt %, 10 wt % and 20 wt % blending concentrations were measured. The ASE thresholds of the films blended with mCP-C6-COT varied between 1.37 and 1.56 μJ cm⁻², which are comparable to that (1.32 μJ cm⁻²) of a BSBCz-EH neat film measured under the same experimental conditions (see FIGS. 10 and 11). The results demonstrate that the use of mCP-C6-COT as a triplet-state quencher additive has a negligible effect on the ASE properties of BSBCz-EH dye.

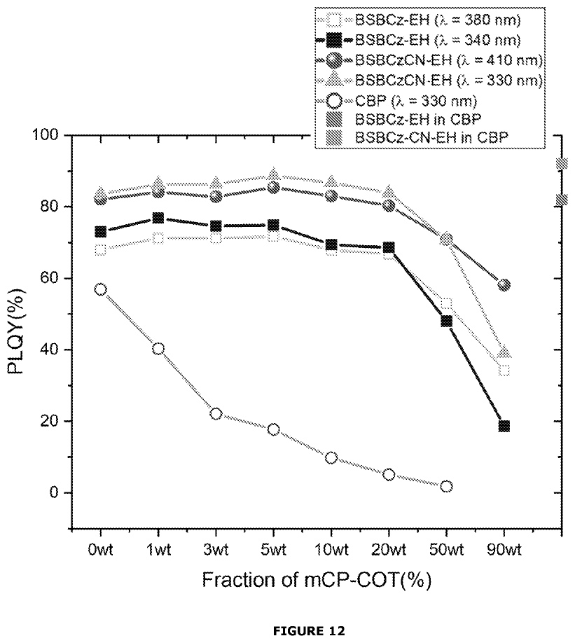

PLQYs of neat and blend-film BSBCz-EH with various mCP-C6-COT (i.e., 1 wt %, 3 wt %, 5 wt %, 10 wt %, 20 wt %, 50 wt % and 90 wt %) are shown in FIG. 12. High PLQY values of 70% were retained for the neat and blend films of BSBCz-EH with up to 20 wt % mCP-C6-COT using excitation wavelength of 380 nm, where only BSBCz-EH was excited. Higher mCP-C6-COT blend concentrations gradually decreased the blend-film PLQYs to 53% and 34% (for the 50 wt % and 90 wt % blends, respectively). Since blend films of BSBCz-EH in common hosts such as CBP showed high PLQYs (Mamada, M., et al. Adv. Funct. Mater. 201828, 1802130), the decrease of blend film PLQYs of BSBCz-EH with mCP-C6-COT can, therefore, be ascribed to the quenching by the COT moiety. However, the quenching seems to be not efficient at moderately high PLQYs even with high COT concentrations (i.e., BSBCz-EH:COT=1:17 mol/mol in the blend film of BSBCz-EH with 90 wt % mCP-C6-COT), probably due to complicated energy transfer processes. It is interesting to note that the decrease in PLQYs with increasing mCP-C6-COT concentration is dependent on the singlet/triplet energy of the emitter. Thus, the higher the singlet/triplet energy of the emitter, the sharper the decrease in PLQYs with increasing mCP-C6-COT concentrations for CBP(S₁=3.5 eV, most quenching), BSBCz-EH (S₁=2.9 eV) and BSBCz-CN-EH (S₁=2.6 eV, least quenching).

Triplet Quenching Studies Under Electrical Excitation:

In order to investigate STA quenching study with electrical excitation in ns pulse width range, a blend of BSBCz-EH with mCP-C6-COT was used to create OLEDs for testing STA quenching and compared with a neat BSBCz-EH OLED device. The structure of small-area OLEDs studied was ITO (100 nm)/PEDOT:PSS (30 nm)/BSBCz-EH (neat or with 2 wt % mCP-C6-COT) (60 nm)/TPBi (40 nm)/LiF (1 nm)/AI (100 nm), where ITO is Indium tin oxide, PEDOT:PSS is poly(3,4-ethylenedioxythiophene)-poly(styrenesulfonate) and TPBi is 1,3,5-tris(2-N-phenylbenzimidazolyl)benzene. The device area of OLEDs was 0.2 mm². To achieve high current densities and high luminance, a fundamental requirement for injection lasing, the small area OLEDs were subjected to pulse widths of 100 ns at voltages varying from 20 to 100 V, where the nanosecond pulse widths also enable limitation of the Joule heating.

Once the nanosecond pulse was applied to the small-area OLEDs, EL signals were generated in the organic emissive layer with a delay resulting from the time needed for holes and electrons to form excitons. Singlet excitons are short-lived and usually have a lifetime of few ns for fluorescent emitters (e.g., 1.4 and 1.5 ns for neat BSBCz-EH and blend BSBCZ-EH with 1 wt % COT, respectively), whereas triplets are slower to reach maximum concentration but are generally orders of magnitude higher in density than singlets due to the longer lifetimes. These non-radiative triplets annihilate singlets causing STA, resulting in higher energy triplets and charge carriers. The evidence of STA can be seen in an EL waveform as a reduction in EL intensity after the initial EL peak within tens of nanoseconds. This drop in intensity depends on the STA rate after which EL intensity achieves a steady state. The higher the STA, the more reduction in EL intensity compared to its peak value.

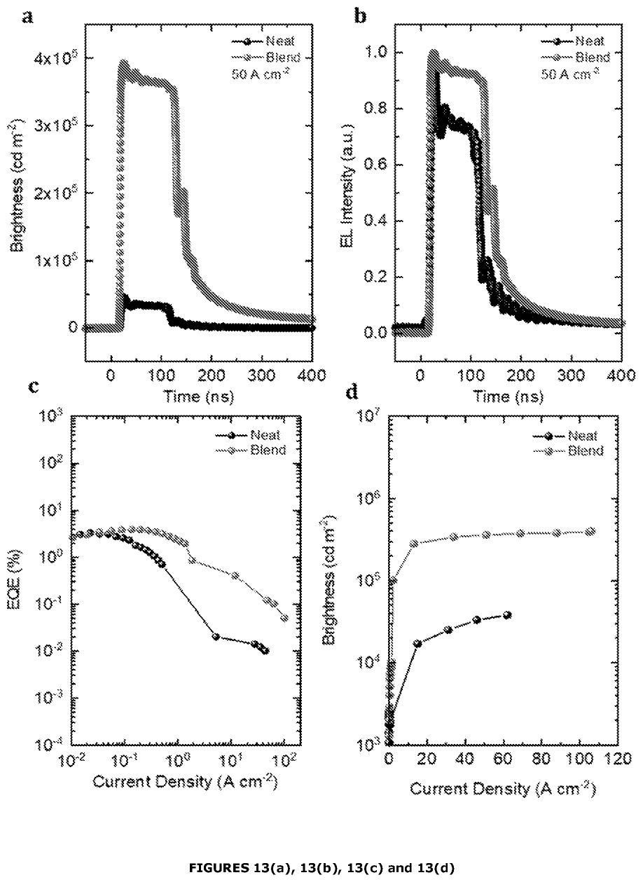

FIG. 13a shows EL response of the neat and blend (with 2 wt % mCP-C6-COT) BSBCz-EH based OLEDs to a 100 ns pulse input, where a considerable reduction in EL intensity can be seen in case of the neat device under the same current density of 50 A cm⁻². FIG. 13b shows normalised EL intensities of the neat and blend OLEDs where a substantial STA can be seen for the neat device. The reduction in intensity after the initial peak was around 25% for the same current. Comparing FIGS. 13a and 13b, it is evident that mCP-C6-COT has aided in reduction of STA. FIGS. 13c and 13d shows plots of EQE and brightness versus current density, presenting a similar order of magnitude improvement in EQEs and brightness.

In order to confirm STA quenching by mCP-C6-COT in the blend films, rate equations for polaron, singlet and triplet generation were simulated in MATLAB® and the STA rate along with other annihilation rates was extracted from the program. Simulation of neat and blend device EL characteristics from rate equations suggests an STA rate (k_{STA}) of 4.3×10⁻⁸ cm³ s⁻¹ for the neat OLEDs. For blend OLEDs, k_{STA} was kept the same and a new term, k_{mCP-C6-COT}, was introduced in the triplet equation depicting contribution of mCP-C6-COT towards rapid triplet depopulation. k_{mCP-C6-COT} was extracted to be 1×10¹⁰ s⁻¹. Singlet density for the blend was found to be around eight times the singlet density in neat device (an indication of more STA quenching in neat device). The results of reduced STA quenching indicate the triplet quencher mCP-C6-COT is efficient for the fast triplet decay. The triplet population obtained for neat devices was found to be almost 30 times more than that of the blend.

The blend OLEDs of BSBCz-EH have been observed to outperform the neat device with EQE reaching close to its theoretical limit of approximately 4% (calculated based on PLQYs of approximately 70% for a fluorescent dye and out-coupling factor of 0.2). The J-V characteristics are also very similar for neat and blend OLEDs depicting no change in electrical behaviour with the addition of mCP-C6-COT in the operating voltage region (>4 V).

The compounds of Formula (I), including mCP-C6-COT, are thus useful as solid-state organic triplet quenchers. It has been demonstrated that mCP-C6-COT is a non-emissive solid-state triplet quencher, exhibiting excellent triplet quenching ability under both optical and electrical excitation, coupled with excellent solution processability.

Photophysical, thermal and electronic properties of mCP-C6-COT demonstrates that the integration of COT and mCP enables mCP-C6-COT to be a solid at room temperature with a high decomposition temperature, and is thus useful for thin film devices and applications. The triplet quenching ability of mCP-C6-COT as an additive was investigated using the solution-processable dye BSBCz-EH under both optical and electrical excitations. Under CW photoexcitation, even small blending concentrations of mCP-C6-COT (equal or less than 20 wt %) showed unprecedented STA suppression. Moreover, over 20-fold improvement in PL stability of BSBCz-EH was found by blending with mCP-C6-COT. Notably, negligible impact on the ASE characteristics of the dye BSBCz-EH was observed as comparable ASE threshold values were measured for the blend films compared to the neat films of BSBCz-EH. STA suppression under electrical excitation in ns regime was demonstrated by employing small area OLED structures. Compared to a BSBCz-EH neat film, a 2 wt % blend of mCP-C6-COT resulted in a substantial reduction of STA observed in the EL-current density curve, coupled with an increase in EL intensity and brightness, comparable EQE maximum, as well as a reduction in EQE roll-off.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A triplet excited-state quencher compound of Formula (I): 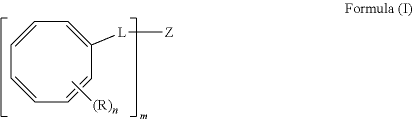
wherein:
Z is a wide band gap moiety;
L is a non-conjugating linker group;
each R, which may be the same or different, is a non-conjugating substituent;
n is an integer from 0 to 7; and
m is an integer from 1 to 6.

2. The compound according to claim 1, wherein the compound has the Formula (Ib): 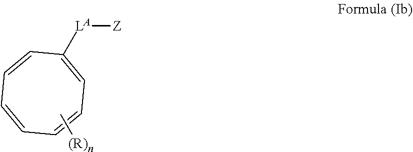
wherein:
Z, R and n are as defined above for a compound of Formula (I); and
L^{A} is a branched or straight chain alkylene linking group comprising two or more carbon atoms; or
L^{A} is —X¹-L^{b}-X²—;
wherein L^{b} is a branched or straight chain alkylene group comprising two or more carbon atoms and X¹ and X² are independently selected from an ether, amino, amide or ester group; and wherein one of X¹ and X² may be absent.

3. The compound according to claim 1, wherein the compound has the Formula (Id): 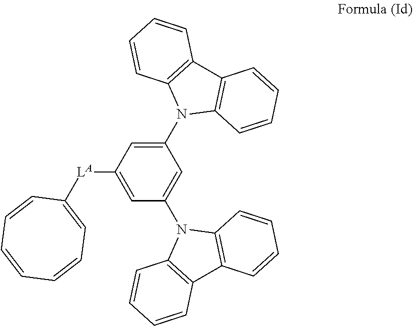
wherein L^{A} is a branched or straight chain alkylene linking group.

4. The compound according to claim 1, wherein the compound is 9,9′-(5-(6-((1Z,3Z,5Z,7Z)-cycloocta-1,3,5,7-tetraen-1-yl)hexyl)-1,3-phenylene)bis(9H-carbazole) (mCP-C6-COT):

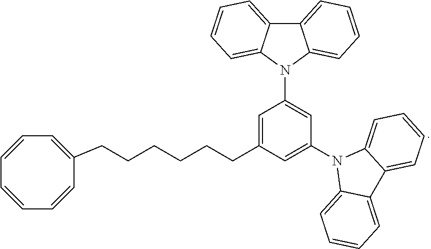 [SMILES: C1=C\C=C/C(CCCCCCc2cc(-n3c4ccccc4c4ccccc43)cc(-n3c4ccccc4c4ccccc43)c2)=C\C=C/1]

5. A use of a compound according to claim 1 as a triplet quencher.

6. A composition comprising a compound according to claim 1 and an organic semiconductor laser dye.

7. A composition according to claim 6 wherein the laser dye is BSBCz-EH or BSBCzCN-EH.

8. The composition according to claim 6, wherein the composition comprises the compound of Formula (I) in an amount of from about 1 wt % to about 20 wt %.

9. The compound according to claim 1, provided as a coating or a thin film.

10. The coating or thin film according to claim 9 provided on a substrate.

11. A film or coating comprising a compound according to claim 1.

12. A use of a composition according to claim 6 as an active gain medium for light amplification.

13. The use according to claim 12, wherein the active gain medium is for light amplification in organic solid-state lasers.

14. The use according to claim 13, wherein the laser is electrically pumped.

15. The use according to claim 12, wherein the composition is for use in at least one of: organic solid-state lasers; opto-electronic applications; laser diodes; light-emitting diodes; solar cells; sensors; and photorefractive devices.
